(19) United States
(12) Patent Application Publication
(10) Pub. No.: US 2026/0058018 A1
(43) Pub. Date: Feb. 26, 2026

(54) METHODS AND SYSTEMS FOR CONFIRMING AN ADVISORY INTERACTION WITH AN ARTIFICIAL INTELLIGENCE PLATFORM

(71) Applicant: KPN INNOVATIONS LLC, LAKEWOOD, CO (US)

(72) Inventor: Kenneth Neumann, LAKEWOOD, CO (US)

(73) Assignee: KPN INNOVATIONS LLC, LAKEWOOD, CO (US)

(21) Appl. No.: 19/314,148

(22) Filed: Aug. 29, 2025

Related U.S. Application Data
(63) Continuation of application No. 17/164,491, filed on Feb. 1, 2021, now Pat. No. 12,417,843.
Application No. 17/164,491 is a continuation-in-part of application No. 17/032,050, filed on Sep. 25, 2020, now Pat. No. 12,087,442.
Application No. 17/032,050 is a continuation-in-part of application No. 16/671,925, filed on Nov. 1, 2019, now Pat. No. 10,936,962.

(51) Int. Cl. G16H 50/20; G06N 20/00; G16H 70/20

(52) U.S. Cl. CPC G16H 50/20; G06N 20/00; G16H 70/20

(57) ABSTRACT

A system for confirming an advisory interaction with an artificial intelligence platform. The system includes a constitutional generator module configured to receive a first advisory input, retrieve an expert input, select a machine-learning process as a function of the expert input, and generate a therapeutic corrector. The system includes a constitutional advisory module configured to display a therapeutic corrector on a graphical user interface and receive a second advisory input. The system includes a best practices module the best practices module designed and configured to retrieve from an expert database a best practices training set, calculate an optimal vector output, generate an optimal vector output containing an expected therapeutic corrector implementation response, authenticate a second advisory input, and update the best practices module.

### CROSS-REFERENCE TO RELATED APPLICATIONS

This application is a continuation-in-part of U.S. Non-provisional application Ser. No. 17/164,491, filed on Feb. 1, 2021 and entitled “METHODS AND SYSTEMS FOR CONFIRMING AN ADVISORY INTERACTION WITH AN ARTIFICIAL INTELLIGENCE PLATFORM,” which is a continuation-in-part of U.S. Non-provisional application Ser. No. 17/032,050, filed on Sep. 25, 2020 and entitled “METHODS AND SYSTEMS FOR CONFIRMING AN ADVISORY INTERACTION WITH AN ARTIFICIAL INTELLIGENCE PLATFORM,” which is a continuation-in-part of U.S. Non-provisional application Ser. No. 16/671,925 filed on Nov. 1, 2019 and entitled “METHODS AND SYSTEMS FOR CONFIRMING AN ADVISORY INTERACTION WITH AN ARTIFICIAL INTELLIGENCE PLATFORM.” Each of U.S. Non-provisional application Ser. No. 17/164,491, Ser. No. 17/032,050 and Ser. No. 16/671,925 are incorporated herein by reference in their entirety.

### FIELD OF THE INVENTION

The present invention generally relates to the field of artificial intelligence. In particular, the present invention is directed to methods and systems for confirming an advisory interaction with an artificial intelligence platform.

### BACKGROUND

Accurate selection and authentication of data entries to be incorporated into an artificial intelligence platform can be challenging. Selection of inaccurate data entries can create data entries that do not produce accurate or informative results. Artificial intelligence (AI) systems, and particularly large language models (LLMs), have demonstrated significant capabilities in processing natural language inputs and generating contextually relevant outputs. However, existing systems often lack robust, automated mechanisms to evaluate and improve the quality of generated outputs in a way that accounts for both expert judgment and objective real-world outcome indicators. There remains a need for an improved AI-based platform that evaluates and improves the quality of generated outputs in real-time.

### SUMMARY OF THE DISCLOSURE

In some aspects, the techniques described herein relate to a system for confirming an advisory interaction with an artificial intelligence platform, the system including at least a processor, and a memory communicatively connected to the at least a processor, wherein the memory contains instructions configuring the at least a processor to receive an advisory input including a constitutional inquiry, generate, using a large language model (LLM) and one or more machine-learning modules, a first therapeutic corrector as a function of the advisory input, wherein the LLM has been trained with first LLM training data including correlations between keywords, receive a feedback input in response to an implementation of the first therapeutic corrector, wherein at least a part of the feedback input indicates that the first therapeutic corrector is incorrect, generate a feedback quality score as a function of the feedback input using a scoring machine-learning model of the one or more machine-learning modules that has been trained with scoring training data including exemplary feedback inputs correlated to exemplary feedback quality scores, update the LLM and the one or more machine-learning modules as a function of at least in part on the feedback quality score, wherein updating includes generating second LLM training data including the first LLM training data and therapeutic correctors that are incorrectly generated using the first LLM training data, and generating a second therapeutic corrector using the updated LLM that is retrained with the second LLM training data, and generate a graphical user interface displaying the second therapeutic corrector.

In some aspects, the techniques described herein relate to a method for confirming an advisory interaction with an artificial intelligence platform, the method including receiving, using at least a processor, an advisory input including a constitutional inquiry, generating, using the at least a processor, a first therapeutic corrector as a function of the advisory input using a large language model (LLM) and one or more machine-learning modules, wherein the LLM has been trained with first LLM training data including correlations between keywords, receiving, using the at least a processor, a feedback input in response to an implementation of the first therapeutic corrector, wherein at least a part of the feedback input indicates that the first therapeutic corrector is incorrect, generating, using the at least a processor, a feedback quality score as a function of the feedback input using a scoring machine-learning model of the one or more machine-learning modules that has been trained with scoring training data including exemplary feedback inputs correlated to exemplary feedback quality scores, updating, using the at least a processor, the LLM and the one or more machine-learning modules as a function of at least in part on the feedback quality score, wherein updating includes generating second LLM training data including the first LLM training data and therapeutic correctors that are incorrectly generated using the first LLM training data, and generating a second therapeutic corrector using the updated LLM that is retrained with the second LLM training data, and generating, using the at least a processor, a graphical user interface displaying the second therapeutic corrector.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for confirming an advisory interaction with an artificial intelligence platform;

FIG. 2 is a block diagram illustrating an exemplary embodiment of an inference model;

FIG. 3 is a block diagram illustrating an exemplary embodiment of a constitutional generator module;

FIG. 4 is a block diagram illustrating an exemplary embodiment of an expert database;

FIG. 5 is a block diagram illustrating an exemplary embodiment of a constitutional advisory module;

FIG. 6 is a block diagram illustrating an exemplary embodiment of an advisor database;

FIG. 7 is a block diagram illustrating an exemplary embodiment of a best practices module;

FIG. 8 is a block diagram illustrating an exemplary embodiment of a user database;

FIG. 9 illustrates a block diagram of an exemplary machine-learning module;

FIG. 10 illustrates a diagram of an exemplary neural network;

FIG. 11 illustrates a block diagram of an exemplary node in a neural network;

FIG. 12 is a process flow diagram illustrating an exemplary embodiment of a method of confirming an advisory interaction with an artificial intelligence platform;

FIG. 13 is a process flow diagram illustrating an exemplary embodiment of another method of confirming an advisory interaction with an artificial intelligence platform;

FIG. 14 is a process flow diagram illustrating an exemplary embodiment of a method of calculating an inference model; and

FIG. 15 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for confirming an advisory interaction with an artificial intelligence platform, the system including at least a processor, and a memory communicatively connected to the at least a processor, wherein the memory contains instructions configuring the at least a processor to receive an advisory input including a constitutional inquiry, generate, using a large language model (LLM) and one or more machine-learning modules, a first therapeutic corrector as a function of the advisory input, wherein the LLM has been trained with first LLM training data including correlations between keywords, receive a feedback input in response to an implementation of the first therapeutic corrector, wherein at least a part of the feedback input indicates that the first therapeutic corrector is incorrect, generate a feedback quality score as a function of the feedback input using a scoring machine-learning model of the one or more machine-learning modules that has been trained with scoring training data including exemplary feedback inputs correlated to exemplary feedback quality scores, update the LLM and the one or more machine-learning modules as a function of at least in part on the feedback quality score, wherein updating includes generating second LLM training data including the first LLM training data and therapeutic correctors that are incorrectly generated using the first LLM training data, and generating a second therapeutic corrector using the updated LLM that is retrained with the second LLM training data, and generate a graphical user interface displaying the second therapeutic corrector.

Exemplary embodiments illustrating aspects of the present disclosure are described below in the context of several specific examples.

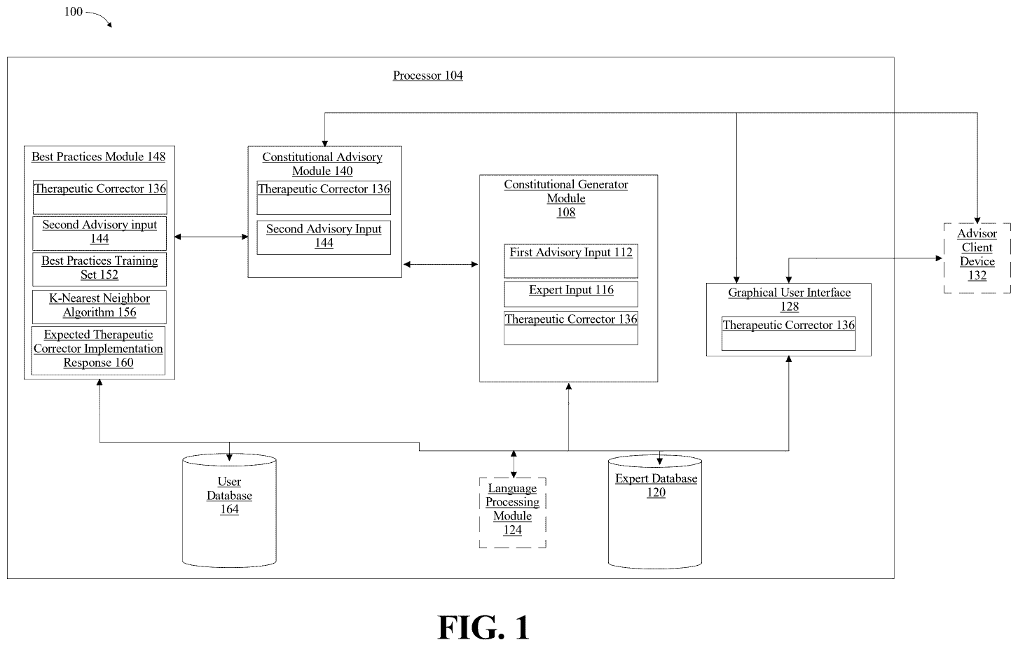

Referring now to the drawings, FIG. 1 illustrates an exemplary embodiment of a system 100 for providing dynamic constitutional guidance. System 100 includes a processor. A processor 104 may include any computing device as described herein, including without limitation a microcontroller, microprocessor, digital signal processor 104 (DSP) and/or system on a chip (SoC) as described herein. A processor 104 may be housed with, may be incorporated in, or may incorporate one or more sensor of at least a sensor. Computing device may include, be included in, and/or communicate with a mobile device such as a mobile telephone or smartphone. A processor 104 may include a single computing device operating independently, or may include two or more computing device operating in concert, in parallel, sequentially or the like; two or more computing devices may be included together in a single computing device or in two or more computing devices. A processor 104 with one or more additional devices as described below in further detail via a network interface device. Network interface device may be utilized for connecting a processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. A processor 104 may include but is not limited to, for example, A processor 104 or cluster of computing devices in a first location and a second computing device or cluster of computing devices in a second location. A processor 104 may include one or more computing devices dedicated to data storage, security, distribution of traffic for load balancing, and the like. A processor 104 may distribute one or more computing tasks as described below across a plurality of computing devices of computing device, which may operate in parallel, in series, redundantly, or in any other manner used for distribution of tasks or memory between computing devices. A processor 104 may be implemented using a “shared nothing” architecture in which data is cached at the worker, in an embodiment, this may enable scalability of system 100 and/or computing device.

With continued reference to FIG. 1, a processor 104 may be designed and/or configured to perform any method, method step, or sequence of method steps in any embodiment described in this disclosure, in any order and with any degree of repetition. For instance, a processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A processor 104 may perform any step or sequence of steps as described in this disclosure in parallel, such as simultaneously and/or substantially simultaneously performing a step two or more times using two or more parallel threads, processor 104 cores, or the like; division of tasks between parallel threads and/or processes may be performed according to any protocol suitable for division of tasks between iterations. A processor 104 may include one or more modules as illustrated herein which demonstrate how a particular system may operate. One or more modules as described in this disclosure may be configured to be implemented as any hardware and/or software module. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, system 100 includes a constitutional generator module 108. Constitutional generator module 108 may be implemented as any hardware and/or software module. Constitutional generator module 108 may be designed and configured to receive a first advisory input 112 containing a constitutional inquiry and a user identifier; retrieve an expert input 116 from an expert database 120 operating on the processor as a function of the first advisory input 112 and the user identifier; select a machine-learning model as a function of the expert input 116; and generate a therapeutic corrector 136 utilizing the machine-learning model and the advisory input wherein the therapeutic corrector 136 further comprises a response to the constitutional inquiry.

With continued reference to FIG. 1, constitutional generator module 108 may be configured to receive a first advisory input 112 containing a constitutional inquiry and a user identifier from an advisor client device operated by an informed advisor. A “first advisory input” as used in this disclosure, includes any medical inquiry generated by an informed advisor. A “medical inquiry” as used in this disclosure, includes any question, input, or advice sought by an informed advisor of a medical nature. Medical nature includes the science and/or practice of the diagnosis, treatment, and prevention of disease. A “constitutional inquiry” as used in this disclosure, includes any inquiry pertaining to the human body. For instance and without limitation, a constitutional inquiry may include advice sought in regard to the best treatment for a user with an aggressive form of cancer. In yet another non-limiting example, a constitutional inquiry may include advice sought in regard to possible diagnoses for a user who complains of symptoms such as chills, body aches, fatigue, and lethargy. An informed advisor, as used in this disclosure, includes a person who is licensed by a state, federal, and/or international licensing agency that helps in identifying, preventing, and/or treating illness and/or disability. An informed advisor may include persons such as a functional medicine doctor, a doctor of osteopathy, a nurse practitioner, a physician assistant, a Doctor of Optometry, a Doctor of Dental Medicine, a Doctor of Dental Surgery, a naturopathic doctor, a Doctor of Physical Therapy, a nurse, a doctor of chiropractic medicine, a doctor of oriental medicine and the like. An informed advisor may include other skilled professionals such as nurses, respiratory therapists, pharmacists, home health aides, audiologists, clinical nurse specialists, nutritionists, dieticians, clinical psychologists, psychiatric mental health nurse practitioners, spiritual coaches, life coaches, holistic medicine specialists, acupuncturests, reiki masters, yoga instructors, holistic health coaches, wellness advisors and the like. An advisor client device may include any device as described below in more detail.

With continued reference to FIG. 1, a “user identifier’ as used in this disclosure, includes any data that uniquely identifies a particular user. Data may include a user's name, a user's date of birth, a user's medical identification number, a public and/or private key pair, a cryptographic hash, a biometric identifier such as an iris scan, fingerprint scan, a palm vein scan, a retina scan, facial recognition, DNA, a personal identification number, a driver's license or passport, token-based identification systems, digital signatures, and the like. A user identifier may be an identifier that is unique as compared to any other user identifier within system 100. A user identifier may include a statistically ensured unique identifier such as a global unique identifier (GUID) or a universally unique identifier (UUID).

With continued reference to FIG. 1, constitutional generator module 108 may be configured to retrieve an expert input 116 from an expert database operating on a processor 104. An “expert input,” as used in this disclosure, includes any expert submission, such as a textual submission, expert paper, form entry or the like. An “expert” as used in this disclosure includes any health professional who may meet one or more criterion including for example obtaining board certification in a particular specialty, having clinical trial experience, being published in textbooks and peer-reviewed medical media, giving presentations at medical meetings, being involved in formulary committee participation, having a thriving clinical practice, affiliations with notation at a teaching institution, treating particular specialties and populations of patients, holding various positions or titles, having a diverse publication history such as prolific authorship, editorials, clinical guidelines and the like, having research experience and the like. A health professional includes any professional suitable for use as an informed advisor. For example, a health professional may include a functional medicine physician or a nurse practitioner who treats heart failure patients. An expert input 116 may include one or more data entries describing current treatment guidelines, best practices for treating a particular disease state, best machine-learning algorithms to generate a response to particular advisory inputs, best machine-learning models to use to generate a response to particular advisory inputs and the like. An expert input 116 may be received live and in real time. In yet another non-limiting example, an expert input 116 may be received at various times and one or more expert input 116 may be stored in an expert database which include any data structure as described in more detail below.

With continued reference to FIG. 1, expert input 116 may include temporal attributes, such as timestamps which may be utilized to select only expert input 116 more recently entered for training data and/or machine-learning model selection as described below in more details. Constitutional generator module 108 may receive an update to one or more expert input 116 and may perform one or more modifications to expert input 116. For example, a clinical trial may turn out to fail and as such constitutional generator module 108 may remove it from data, as a result. In yet another non-limiting example, a medical and/or academic paper, or a study on which it was based, may be revoked; and constitutional generator module 108 may remove it. Expert input 116 may be stored in one or more database structures on best practices module 148 operating on a processor 104.

With continued reference to FIG. 1, expert input 116 may be stored in an expert database 120 located within best practices module. Expert database 120 may be implemented, without limitation, as a relational database, a key-value retrieval datastore such as a NOSQL database, or any other format or structure for use as a datastore that a person skilled in the art would recognize as suitable upon review of the entirety of this disclosure. Expert input 116 may include textual data such as numerical, character, and/or string data. Textual data may include a standardized name and/or code for a disease, disorder, measurement, or the like; codes may include diagnostic codes and/or diagnosis codes, which may include without limitation codes used in diagnosis classification systems such as The International Statistical Classification of Diseases and Related Health Problems (ICD). In general, there is no limitation on forms textual data or non-textual data used expert input 116 may take; persons skilled in the art, upon reviewing the entirety of this disclosure, will be aware of various forms which may be suitable for use as periodic longevity consistently with this disclosure.

With continued reference to FIG. 1, expert database 120 may store one or more expert input 116 as image data, such as for example, a computed tomography (CT) scan or a magnetic resonance image (MRI). Image data may be stored in various forms including for example, joint photographic experts group (JPEG), exchangeable image file format (Exif), tagged image file format (TIFF), graphics interchange format (GIF), portable network graphics (PNG), netpbm format, portable bitmap (PBM), portable any map (PNM), high efficiency image file format (HEIF), still picture interchange file format (SPIFF), better portable graphics (BPG), drawn filed, enhanced compression wavelet (ECW), flexible image transport system (FITS), free lossless image format (FLIF), graphics environment manage (GEM), portable arbitrary map (PAM), personal computer exchange (PCX), progressive graphics file (PGF), gerber formats, 2 dimensional vector formats, 3 dimensional vector formats, compound formats including both pixel and vector data such as encapsulated postscript (EPS), portable document format (PDF), and stereo formats.

With continued reference to FIG. 1, system 100 may include a language processing module 124. Language processing module 124 may be configured to extract one or more words from a first advisory input 112 and/or a user identifier and retrieve an expert input 116 based on advisory input and/or the user identifier. Language processing module 124 may include any hardware and/or software module. Language processing module 124 may be configured to extract, from one or more inputs, one or more words. One or more words may include, without limitation, strings of one or characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model.

With continued reference to FIG. 1, language processing module 124 may operate to produce a language processing model. Language processing model may include a program automatically generated by a processor 104 and/or language processing module 124 to produce associations between one or more words extracted from at least a document and detect associations, including without limitation mathematical associations, between such words, and/or associations of extracted words with categories of physiological data, relationships of such categories to prognostic labels, and/or categories of prognostic labels. Associations between language elements, where language elements include for purposes herein extracted words describing and/or including constitutional data and/or ameliorative recommendation data may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of physiological data, a given relationship of such categories to prognostic labels, and/or a given category of prognostic labels. As a further example, statistical correlations and/or mathematical associations may include probabilistic formulas or relationships indicating a positive and/or negative association between at least an extracted word and/or a given element of constitutional data and/or ameliorative recommendation data; positive or negative indication may include an indication that a given document is or is not indicating an element of constitutional data and/or ameliorative recommendation data. For instance, and without limitation, a negative indication may be determined from a phrase such as “telomere length was not found to be an accurate predictor of overall longevity,” whereas a positive indication may be determined from a phrase such as “telomere length was found to be an accurate predictor of dementia,” as an illustrative example; whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory on a processor 104, or the like.

Still referring to FIG. 1, language processing module 124 and/or a processor 104 may generate a language processing model by any suitable method, including without limitation a natural language processing classification algorithm; language processing model may include a natural language process classification model that enumerates and/or derives statistical relationships between input term and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs as used herein are statistical models with inference algorithms that may be applied to the models. In such models, a hidden state to be estimated may include an association between an extracted word category of physiological data, a given relationship of such categories to prognostic labels, and/or a given category of prognostic labels. There may be a finite number of category of physiological data, a given relationship of such categories to prognostic labels, and/or a given category of prognostic labels to which an extracted word may pertain; an HMM inference algorithm, such as the forward-backward algorithm or the Viterbi algorithm, may be used to estimate the most likely discrete state given a word or sequence of words. Language processing module 124 may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Continuing to refer to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

Still referring to FIG. 1, language processing module 124 may use a corpus of documents to generate associations between language elements in a language processing module 124, and a processor 104 may then use such associations to analyze words extracted from one or more documents and determine that the one or more documents indicate significance of a category of physiological data, a given relationship of such categories to prognostic labels, and/or a given category of prognostic labels. In an embodiment, a processor 104 may perform this analysis using a selected set of significant documents, such as documents identified by one or more experts as representing good science, good clinical analysis, or the like; experts may identify or enter such documents via a graphical user interface 128 as described below, or may communicate identities of significant documents according to any other suitable method of electronic communication, or by providing such identity to other persons who may enter such identifications into a processor 104. Documents may be entered into a processor 104 by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, a processor 104 may automatically obtain the document using such an identifier, for instance by submitting a request to a database or compendium of documents such as JSTOR as provided by Ithaka Harbors, Inc. of New York.

With continued reference to FIG. 1, system 100 may include a graphical user interface 128. Graphical user interface 128 may include without limitation a form or other graphical element having data entry fields, wherein one or more experts, including without limitation clinical and/or scientific experts, may enter information describing one or more expert input 116. Fields in graphical user interface 128 may provide options describing previously identified submissions including, for instance drop-down lists where experts may be able to select one or more entries to indicate their usefulness and/or significance in the opinion of the experts. Fields may include free-form entry fields such as text-entry fields where an expert may be able to type or otherwise enter text, enabling an expert to propose or suggest new or amended inputs not previously recorded. Graphical user interface 128 may include fields corresponding to machine-learning models, training sets, and/or machine-learning algorithms where an expert may view particular diagrams and/or pictures of any of the above. Graphical user interface 128 may allow for an expert to zoom in on a particular field or open a drop-down list in a new window to highlight more details. Graphical user interface 128 may include a field that allows an expert to indicate a reference to a particular document or journal article.

With continued reference to FIG. 1, one or more expert input 116 may be received from an advisor client device. Advisor client device 132 may include without limitation, a display in communication with a processor, where a display may include any display as described herein. Advisor client device 132 may include an additional computing device, such as a mobile device, laptop, desktop computer and the like. Advisor client device 132 may transmit one or more expert input 116 to processor 104 utilizing any network methodology as described herein. Advisor client device may be operated by an informed advisor. An informed advisor may include any of the informed advisors as described herein.

With continued reference to FIG. 1, constitutional generator module 108 may be configured to select a machine-learning process as a function of an expert input 116. A machine learning process is a process that automatedly uses a body of data known as “training data” and/or a “training set” to generate an algorithm that will be performed by a computing device/module to produce outputs given data provided as inputs; this is in contrast to a non-machine learning software program where the commands to be executed are determined in advance by a user and written in a programming language.

With continued reference to FIG. 1, supervised machine-learning algorithms, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may use elements of comprehensive diagnoses as inputs, priority treatments as outputs, and a scoring function representing a desired form of relationship to be detected between elements of comprehensive diagnoses and priority treatments; scoring function may, for instance, seek to maximize the probability that a given element of a comprehensive diagnosis is associated with a given priority treatment and/or combination of comprehensive diagnoses to minimize the probability that a given element of a comprehensive diagnosis and/or combination of elements comprehensive diagnoses are not associated with a given priority treatment and/or combination of priority treatments. Scoring function may be expressed as a risk function representing an “expected loss” of an algorithm relating inputs to outputs, where loss is computed as an error function representing a degree to which a prediction generated by the relation is incorrect when compared to a given input-output pair provided in a training set. Persons skilled in the art, upon reviewing the entirety of this disclosure, will be aware of various possible variations of supervised machine-learning algorithms that may be used to determine relation between comprehensive diagnoses and priority treatments. In an embodiment, one or more supervised machine-learning algorithms may be restricted to a particular domain for instance, a supervised machine-learning process may be performed with respect to a given set of parameters and/or categories of parameters that have been suspected to be related to a given set of comprehensive diagnoses, and/or are specified as linked to a medical specialty and/or field of medicine covering a particular body system or medical specialty. As a non-limiting example, a particular set of diagnoses that indicate emergency medical conditions may be typically associated with a known urgency to seek medical attention and be treated, and a supervised machine-learning process may be performed to relate those comprehensive diagnoses to priority treatments; in an embodiment, domain restrictions of supervised machine-learning procedures may improve accuracy of resulting models by ignoring artifacts in training data. Domain restrictions may be suggested by experts and/or deduced from known purposes for particular evaluations and/or known tests used to evaluate priority treatments. Additional supervised learning processes may be performed without domain restrictions to detect, for instance, previously unknown and/or unsuspected relationships between comprehensive diagnoses and priority treatments.

With continued reference to FIG. 1, “training data,” as used in this disclosure, is data containing correlation that a machine-learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning processes as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), enabling processes or devices to detect categories of data.

Alternatively or additionally, and still referring to FIG. 1, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below. Training data used by a processor may correlate any input data as described in this disclosure to any output data as described in this disclosure.

With continued reference to FIG. 1, a machine-learning process may include an unsupervised machine-learning process. An unsupervised machine-learning process, as used herein, is a process that derives inferences in datasets without regard to labels; as a result, an unsupervised machine-learning process may be free to discover any structure, relationship, and/or correlation provided in the data. An unsupervised machine-learning process may include calculating one or more algorithms or equations including clustering algorithms such as hierarchical clustering, k-means clustering, mixture models, DBSCAN, OPTICS algorithm, and the like; anomaly detection such as local outlier factor; neural networks such as autoencoders, deep belief nets, Hebbian learning, generative adversarial networks, self-organizing map, and the like.

With continued reference to FIG. 1, a machine-learning process may include a lazy-learning process. A lazy-learning process and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover a “first guess” at generating a therapeutic corrector 136. As a non-limiting example, an initial heuristic may include a ranking of potential treatments according to relation to a test type of a first advisory input 112; ranking may include, without limitation, ranking according to significance scores of associations between of a first advisory input 112 and potential treatments, for instance as calculated as described above. Heuristic may include selecting some number of highest-ranking associations and/or potential treatments. Constitutional generator module 108 may alternatively or additionally implement any suitable “lazy learning” algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate therapeutic corrector 136 outputs as described in this disclosure, including without limitation lazy learning applications of machine-learning algorithms as described in further detail below.

With continued reference to FIG. 1, unsupervised processes may be subjected to domain limitations. For instance, and without limitation, an unsupervised process may be performed regarding a comprehensive set of data regarding one person, such as demographic information including age, sex, race, geographical location, profession, and the like. As another non-limiting example, an unsupervised process may be performed on data concerning a particular cohort of persons; cohort may include, without limitation, a demographic group such as a group of people having a shared age range, ethnic background, nationality, sex, and/or gender. Cohort may include, without limitation, a group of people having a shared value for an element and/or category of dietary data, a group of people having a shared value for an element and/or category of demographic data; as illustrative examples, cohort could include all advisory inputs relating to diagnoses, all advisory inputs relating to treatments, all advisory inputs relating to suggested laboratory results or the like. Persons skilled in the art, upon reviewing the entirety of this disclosure, will be aware of a multiplicity of ways in which cohorts and/or other sets of data may be defined and/or limited for a particular unsupervised learning process.

With continued reference to FIG. 1, constitutional generator module 108 may be configured to generate a therapeutic corrector 136 utilizing the machine-learning process and the advisory input. A “therapeutic corrector” as used in this disclosure, includes any response generated in response to an inquiry contained within a constitutional inquiry. A response may include a list of potential diagnoses, a list of available treatment options to try, a list of suggested lab work to analyze, and the like. For instance and without limitation, a therapeutic inquiry may contain a user's lab work showing elevate sodium levels and positive b-type natriuretic peptide and contain an inquiry looking for a list of potential diagnoses. Constitutional generator module 108 may utilize any machine-learning process as described above to generate a therapeutic corrector 136 that includes a list of potential diagnoses that includes heart failure, myocardial infarction, and chronic fatigue syndrome. In yet another non-limiting example, a therapeutic inquiry may contain an inquiry that includes a recommendation for potential lab results that a functional medicine physician should run for a user who complains of symptoms such as weight loss, fatigue, depression, and lack of concentration. Constitutional generator module 108 may utilize any machine-learning process as described above to generate a therapeutic corrector 136 that includes a list of suggested lab results that includes enzyme linked immunosorbent assay (ELISA) for Lyme disease, Vitamin D panel, and a complete thyroid panel. Generating a therapeutic corrector 136 may include receiving therapeutic training data from an expert database 120 wherein the therapeutic training data includes a plurality of data entries containing constitutional inquiries correlated to therapeutic corrector 136; and generating using a supervised machine-learning algorithm a therapeutic model that outputs a therapeutic corrector 136 utilizing the therapeutic training data and the advisory input containing the constitutional inquiry. Therapeutic training data may include any of the training data as described above. In an embodiment, an expert input 116 may be utilized to select a particular training data set based on a particular constitutional inquiry. Generating a therapeutic corrector 136 may include receiving a plurality of unclassified data entries from an expert database 120; and generating using an unsupervised machine-learning algorithm an unsupervised model that outputs a therapeutic corrector 136 utilizing the plurality of unclassified data entries and the advisory input containing the constitutional inquiry.

With continued reference to FIG. 1, system 100 includes a constitutional advisory module 140. A constitutional advisory module 140 may be implemented as any hardware and/or data structure. A constitutional advisory module 140 is designed and configured to receive the therapeutic corrector 136 from the constitutional generator module 108; display the therapeutic corrector 136 on a graphical user interface 128 located on the processor; and receive a second advisory input 144 from an advisor client device operated by an informed advisor wherein the second advisory input contains a therapeutic corrector implementation response.

With continued reference to FIG. 1, a “second advisory input” as used in this disclosure, includes any input generated by an informed advisor in response to a therapeutic corrector. A second advisory input 144 includes a therapeutic corrector implementation response. A “therapeutic corrector implementation response” as used in this disclosure, includes any description that describes an informed advisor's experience with implementing or not implementing a particular therapeutic corrector 136. Implementation may include any effort that an informed advisor may put forth in regard to utilizing a particular therapeutic corrector 136 in the informed advisor's clinical practice and in reference to the particular user that the therapeutic corrector 136 was generated in reference to. A therapeutic implementation response may contain a description that describes how much or how little an informed advisor implemented a particular therapeutic corrector 136 and may also describe whether the therapeutic corrector 136 ultimately helped the user. A therapeutic implementation response may contain a description of one or more results an informed advisor noticed based on implementing a particular therapeutic corrector. Results may include side effects, changes in lab values, subsequently generated diagnoses, elimination of one or more diagnoses, addition of one or more diagnoses, additional tests performed, additional clinical lab work performed and the like. For instance and without limitation, an informed advisor may generate a therapeutic corrector implementation response in reference to a therapeutic corrector 136 that contained a list of suggested laboratory tests to consider in order to help diagnose a user with mysterious symptoms. In such an instance, the informed advisor may generate a therapeutic corrector implementation response that contains a description of which of the suggested laboratory tests the informed advisor performed and if any aided the informed advisor in determining a diagnosis for the user. In yet another non-limiting example, an informed advisor may generate a therapeutic corrector implementation response in reference to a therapeutic corrector 136 that contained a list of two suggested treatment options to consider for a user that the informed advisor had diagnosed as having advanced multiple sclerosis. In such an instance, an informed advisor may generate a therapeutic corrector implementation response that contains a description of the user's experience with trying the two suggested treatment options and if either of the two treatment options helped slow the progression of the advanced multiple sclerosis. A therapeutic corrector implementation response may contain a description that details that the informed advisor chooses not to implement any of the suggested therapeutic corrector 136.

With continued reference to FIG. 1, constitutional advisory module 140 may be configured to authenticate advisory inputs. Constitutional advisory module 140 may receive in conjunction with a therapeutic corrector implementation response a first expert credential validator. A “first expert credential validator” as used in this disclosure, includes any unique identifier that validates a particular user as being an expert. A unique identifier may include an identifier that is unique to system 100, such as a series of numbers and/or letters. A unique identifier may include one or more licensing credentials such as a national provider identifier (NPI), a drug enforcement agency (DEA) number, an institutional provider identifier, a state licensing credential, and the like. Constitutional advisory module 140 may compare the first expert credential validator to a list of known expert credentials stored in an expert database 120. A list of known expert credentials may include a list of all known experts and expert credentials stored within expert database 120. A list of known expert credentials may be updated in live time to account for experts who may have one or more credentials taken away for misconduct, expire, lapse, gain new credentials and the like. Constitutional advisory module 140 determines that the first expert credential validator is authentic by confirming that the first expert credential validator is contained within the list of known expert credentials. Constitutional advisory module 140 authenticates the first advisory input 112 as a function of determining that the first expert credential validator is authentic.

With continued reference to FIG. 1, system 100 includes a best practices module 148. Best practices module 148 may be implemented as any hardware and/or software module. Best practices module 148 may be designed and configured to receive the second advisory input 144 containing the therapeutic corrector implementation response from the constitutional advisory module 140; receive the therapeutic corrector 136 from the constitutional generator module 108; retrieve from an expert database 120 located on the processor a best practices training set 152 wherein the best practices training set 152 correlates a therapeutic corrector 136 to therapeutic corrector implementation responses; calculate an optimal vector output for the therapeutic corrector 136 received from the constitutional generator module 108; generate an optimal vector output containing an expected therapeutic corrector implementation response 160; authenticate the advisory input containing the therapeutic corrector implementation response as a function of the expected therapeutic corrector implementation response 160; and update the best practices module 148 as a function of authenticating the second advisory input 144 containing the therapeutic corrector implementation response to the expected therapeutic corrector implementation response 160.

With continued reference to FIG. 1, best practices training set 152 may implemented as any training data as described above. Best practices training set 152 may include a plurality of data entries correlating a therapeutic corrector 136 to a therapeutic corrector implementation response. For instance and without limitation, best practices training set 152 may correlate a therapeutic corrector 136 such as implementation of low dose naltrexone (LDN) for chronic fatigue syndrome to a therapeutic corrector implementation response that includes increased energy, decreased fatigue, and greater ability to concentrate. In yet another non-limiting example, best practices training set 152 may correlate a therapeutic corrector 136 such as a diagnosis of rheumatoid arthritis to contain a therapeutic corrector implementation response that includes decreased joint pain, and better ability to exercise.

With continued reference to FIG. 1, best practices module 148 may be configured to calculate an optimal vector output for the therapeutic corrector 136 utilizing a k-nearest neighbor algorithm 156 and best practices training set 152. “Optimal vector output” as used in this disclosure, includes a “first guess” by best practices module at the nearest vector in the feature space containing an expected therapeutic corrector implementation response. An “expected therapeutic corrector implementation response” as used in this disclosure, includes any probable or predictable response to implementing a particular therapeutic corrector. Probable or predictable response may be known based on currently available medical literature, case studies, journal articles, expert input, data aggregations from surveyed responses, and the like. For instance and without limitation, a therapeutic corrector 136 such as initiating a fitness regimen may be related to an expected therapeutic corrector implementation response 160 such as weight loss. A therapeutic corrector 136 may be related to one or more expected therapeutic corrector implementation response 160. For instance and without limitation, a therapeutic corrector 136 such as diagnosis of generalized anxiety disorder may be related to one or more expected therapeutic corrector implementation response 160 that include decreased number of anxiety attacks, increased sleep, decreased chest pain, decreased anxiety caused by receiving a medical diagnosis, increased confidence, and the like. In yet another non-limiting example, a therapeutic corrector 136 such as a series of suggested lab panels and medical tests may be related to one or more expected therapeutic corrector implementation response 160 that include absence or presence of expected results, further lab tests or medical tests that were ordered, a potential diagnosis generated based on the suggested lab panels, and the like. K-nearest neighbor algorithm 156 may return a single matching entry or a plurality of matching entries. When a plurality of matching entries is returned, best practices module 148 may derive optimal vector from plurality of matching entries by aggregating matching entries; aggregation may be performed using any suitable method for aggregation, including component-wise addition followed by normalization, component-wise calculation of arithmetic means, or the like. “K-nearest neighbor algorithm 156” as used in this disclosure, includes a lazy-learning method that utilizes feature similarity to analyze how closely out-of-sample-features resemble training data to locate possible optimal vector output, classify possible optimal vector output, calculate an optimal vector output, and generate an optimal vector output. Calculating an optimal vector output utilizing a k-nearest neighbor algorithm 156 may include specifying a K-value, selecting k entries in a database which are closest to the known sample, determining the most common classifier of the entries in the database, and classifying the known sample. A lazy-learning process and/or protocol, which may alternatively be referred to as a “lazy loading” or “call-when-needed” process and/or protocol, may be a process whereby machine-learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data. Heuristic may include selecting some number of highest-ranking associations and/or training data elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

With continued reference to FIG. 1, best practices module 148 authenticates an advisory input containing a therapeutic corrector implementation response as a function of the expected therapeutic corrector implementation response 160. Best practices module 148 may compare a therapeutic corrector implementation response to one or more expected therapeutic corrector implementation response 160 to determine if the therapeutic corrector implementation response matches one or more expected therapeutic corrector implementation response 160. A therapeutic corrector implementation response that does not match one or more expected therapeutic corrector implementation response 160 may require further investigation and authentication. In such an instance, best practices module 148 may authenticate an advisory input containing a therapeutic corrector implementation response that does not match an expected therapeutic corrector implementation response 160 by obtaining a second opinion from a second informed advisor. Best practices module 148 may display the advisory input containing the therapeutic corrector implementation response and the expected therapeutic corrector implementation response 160 on a graphical user interface 128 located on the processor to a second informed advisor. A second informed advisor may include any other informed advisor other than a first informed advisor. A second informed advisor may include an expert in a particular field or specialty that may be related to a particular therapeutic corrector 136. In an embodiment, an informed advisor's area of expertise or specialty may be contained within expert database 120 and may be stored on list of experts. Best practices module 148 receives a second expected therapeutic corrector implementation response 160 from the second informed advisor. A “second expected therapeutic corrector implementation response 160” as used in this disclosure, includes any response suitable for use as first expected therapeutic corrector implementation response 160. A second expected therapeutic corrector implementation response 160 may be generated by an informed advisor who may be an expert or specialist in a particular field, specialty, and/or sub-specialty of medicine. In an embodiment, a second expected therapeutic corrector implementation response 160 may contain a response that may authenticate or not authenticate a response contained within a therapeutic corrector implementation response. For example, a therapeutic corrector implementation response may contain a description of testing for heavy metals in a user that showed user had high levels of cadmium which a first informed advisor attributed to excessive cigarette smoking by the user. A second expected therapeutic corrector implementation response 160 generated by a second informed advisor who may be a specialist in the field of toxicology may authenticate cadmium toxicity due to excessive cigarette smoking when an expected therapeutic implementation response does not contain cadmium toxicity due to cigarette smoking. Best practices module 148 may authenticate a second informed advisor's credentials and expertise in a fashion similar to authenticating a first informed advisor's credentials. Best practices module 148 may receive a second expert credential validator. Second expert credential validator may include any expert credential validator suitable for use as first expert credential validator. Best practices module 148 may compare the second expert credential validator to a list of known expert credentials storied in the expert database 120. Best practices module 148 may determine that the second expert credential validator is authentic and authenticate the second advisory input 144 as a function of determining that the second expert credential validator is authentic.

With continued reference to FIG. 1, best practices module 148 may authenticate an advisory input containing a therapeutic implementation response utilizing an expert periodical submission. An “expert periodical submission” as used in this disclosure, includes any publication written by one or more experts. A publication may include a journal article, clinical trial results, clinical trial data, a news article, a review, academic articles, scholarly articles and the like. Best practices module 148 may retrieve an expert periodical submission contained within the expert database 120. Best practices module 148 may locate an expected therapeutic corrector implementation response 160 contained within an expert periodical. For example, a particular journal article may describe an n of 1 study where an informed advisor observed a particular reaction to a medication only given to one user. Best practices module 148 may located this expected therapeutic corrector implementation response 160 contained within the journal article and compare it to a therapeutic corrector implementation response received from a first informed advisor who may have observed the same reaction to the medication. In an embodiment, expert periodical submissions may be organized within a best practices module 148 by topic to allow for easy searching and indexing to find expert periodical submissions that may pertain to a particular therapeutic corrector implementation response. Best practices module 148 may confirm the legitimacy of the first therapeutic implementation response when it finds the first therapeutic implementation response in an expert periodical submission located within expert database 120. Best practices module 148 may authenticate an advisory input by evaluating a user response to a particular therapeutic corrector 136. Best practices module 148 may retrieve an element of user constitutional data from a user database 164. “User constitutional data” as used in this disclosure, includes any data describing any health process and/or health measurement of a user. A health process may include any treatment prescribed for a user. Treatments may include any ameliorative process including prescription medications, medical procedures, medical tests, medical diagnostics, nutraceuticals, supplements, homeopathic remedies, exercise regimen, fitness routine, yoga practice, meditation sequence, relaxation techniques and the like. A health measurement may include any physically extracted sample which includes a sample obtained by removing and analyzing tissue and/or fluid. Physically extracted sample may include without limitation a blood sample, a tissue sample, a buccal swab, a mucous sample, a stool sample, a hair sample, a fingernail sample, or the like. User constitutional data may include one or more user generated responses detailing how a user feels after implementing a particular health process and/or having a particular health measurement analyzed. One or more user generated responses may be stored in a user database 164. User database 164 may be implemented as any data structure suitable for use as expert database 120 as described above. A user generated response may contain a description of how a user feels after implementing a particular meditation sequence and if the meditation sequence is helping treatment a particular ailment. Best practices module 148 may retrieve a particular element of user constitutional data and compare the element of user constitutional data to a therapeutic corrector implementation response. Best practices module 148 may then authenticate a first therapeutic implementation response utilizing an element of user constitutional data. For example, an element of user constitutional data that describes a user response to a medication as improving the user's fatigue may be utilized to authenticate a first therapeutic implementation response that contains a description of decreased fatigue from the medication user was taking which was a previously unreported side effect of the medication. In yet another non-limiting example, an element of user constitutional data that contains a user's chem-7 panel may be utilized to authenticate a first therapeutic implementation response that contains a description of increased serum sodium levels observed during exercise. In such an instance, best practices module 148 may evaluate a user's serum sodium level collected during exercise to confirm that it was increased. This may be performed when other methods of authentication are not available such as when increased serum sodium levels are not found in any expert periodical submissions or are unable to be authenticated by a second informed advisor.

With continued reference to FIG. 1, best practices module 148 updates the best practices module 148 as a function of authenticating the first advisory input 112 containing the therapeutic corrector implementation response. Updating the best practices module 148 may include incorporating a therapeutic corrector implementation response and/or a therapeutic corrector 136 into the best practices module 148 and/or expert database 120. For example, best practices module 148 may incorporate the therapeutic corrector 136 and the therapeutic corrector implementation response into a best practice training set 152. Updating the best practices module 148 may include incorporating the machine-learning model into the best practices module 148. A second advisory input 144 containing a therapeutic corrector implementation response that does not get authenticated may not be updated into the best practices module 148 so that non-authenticated responses are not utilized to generate subsequent responses within system 100. Instead, non-authenticated responses may be discarded and may not be incorporating into the best practices module 148.

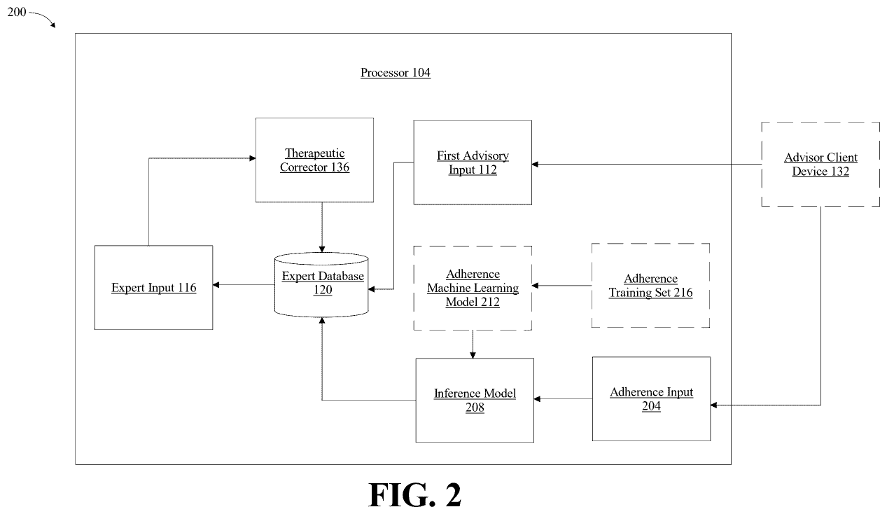

Referring now to FIG. 2, an exemplary embodiment 200 of an inference model is illustrated. In some cases, processor 104 may be configured to obtain an adherence input 204 from advisor client device 132. As used in this disclosure an “adherence input” is an input that represents a user's adherence to therapeutic corrector. For example, and without limitation, therapeutic corrector 136 of 7 days of exercise may relate to adherence input 204 of 4 days. Additionally or alternatively, adherence input 204 may include a quantitative value associated with the therapeutic corrector 136. For example, and without limitation, adherence input may be 22, wherein 22 represents a percentage of completion for therapeutic corrector 136. As a further non-limiting example, adherence input 204 may include a quantitative value of 6 for the number of completed tasks that therapeutic corrector 136 generated. Adherence input 204 may relate to one or more monitoring devices that are capable of monitoring a user's physiological data. As used in this disclosure “monitoring device” is an electronic device that is worn on the person of a user, such as without limitation close to and/or on the surface of the skin, wherein the device can detect, analyze, and transmit biochemical information concerning an individual. Monitoring device may include, without limitation, any device that further collects, stores, and analyzes data associated with adherence input 204. Monitoring device my consist of, without limitation, near-body electronics, on-body electronics, in-body electronics, electronic textiles, smart watches, smart glasses, smart clothing, fitness trackers, body sensors, wearable cameras, head-mounted displays, body worn cameras, Bluetooth headsets, wristbands, smart garments, chest straps, sports watches, fitness monitors, and the like thereof. Monitoring device may include, without limitation, earphones, earbuds, headsets, bras, suits, jackets, trousers, shirts, pants, socks, bracelets, necklaces, brooches, rings, jewelry, AR HMDs, VR HMDs, exoskeletons, location trackers, and gesture control wearables.

Still referring to FIG. 2, processor 104 may be configured to obtain adherence input 204 by determining a therapeutic effect as a function of therapeutic corrector 136. As used in this disclosure a “therapeutic effect” is the physiological response of the user's body due to therapeutic corrector 136. As a non-limiting example a therapeutic response of reduced inflammation in a particular organ and/or tissue as a result of a therapeutic corrector associated with antioxidants. As a further non-limiting example, a therapeutic response of increased catabolic function may be a result of a therapeutic corrector associated with increased piperine. Therapeutic effect may be determined by establishing a therapeutic enumeration. As used in this disclosure a “therapeutic enumeration” is a measurable value associated with a user's symptoms. As a non-limiting example, a therapeutic enumeration of 23 may be identified for a user's elevated levels of saturated fats. As a further non-limiting example, a therapeutic enumeration of 7 may be identified as a function of a decreased caloric expenditure. Therapeutic effect may be determined by distinguishing a therapeutic divergence. As used in this disclosure a “therapeutic divergence” is a measurable value comprising the magnitude of divergence of therapeutic enumeration and a therapeutic recommendation. As used in this disclosure a “therapeutic recommendation” is a recommendation and/or guideline associated with the user's symptoms. As a non-limiting example, therapeutic recommendation recommend that a user produces 1.42 Liters and/or 1.5 quarts of mucus per day, wherein a therapeutic divergence may be 2 for a user that produces 3.87 Liters and/or 4.09 quarts of mucus per day. Therapeutic effect may be determined as a function of therapeutic enumeration, therapeutic divergence, and a therapeutic threshold. As used in this disclosure a “therapeutic threshold” is an upper and/or lower limit that a therapeutic divergence should not diverge from. For example, and without limitation, therapeutic threshold may include an upper limit of 8, wherein a lower limit is 3, for a therapeutic divergence associated with O₂ saturation. As a further non-limiting example, therapeutic threshold may include an upper limit of 23, wherein a lower limit is 13, for a therapeutic divergence associated with bacterial presence in the blood.

Still referring to FIG. 2, processor 104 may be configured to calculate an inference model 208. As used in this disclosure an “inference model” is an algorithm and/or function that produces associations between one or more adherence inputs such that a prediction and/or estimation of the next user's symptoms and or progression. As a non-limiting example, inference model 208 may include an algorithm for the prediction of a particular symptom and/or illness. For example, and without limitation, therapeutic corrector 136 may identify a treatment for COVID-19, wherein inference model 208 may be calculated to predict the next symptoms and/or therapeutic correctors that the user may require. Inference model 208 may be calculated as a function of an adherence machine-learning model 212. As used in this disclosure “adherence machine-learning model” is a machine-learning model to produce an inference model given adherence inputs as inputs; this is in contrast to a non-machine learning software program where the commands to be executed are determined in advance by a user and written in a programming language. Adherence machine-learning model 212 may include one or more adherence machine-learning processes such as supervised, unsupervised, or reinforcement machine-learning processes that processor 104 and/or a remote device may or may not use in the determination of inference model 208. As used in this disclosure “remote device” is an external device to processor 104. An adherence machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 2, processor 104 may train adherence machine-learning model 212 as a function of an adherence training set 216. As used in this disclosure “adherence training set” is a training set that correlates a monitoring element and/or a therapeutic corrector to an inference model. As used in this disclosure a “monitoring element” is an element of datum that is identified as a function of a monitoring device, wherein a monitoring device is described above in detail. For example, and without limitation, a monitoring element of elevated heart rate and a therapeutic corrector of a reduced heart rate may relate to an inference model that calculates the predicted next symptoms and/or effects. Adherence training set 216 may be received as a function of user-entered valuations of monitoring elements, therapeutic correctors, and/or inference models. Processor 104 may receive adherence training set by receiving correlations of monitoring elements and/or therapeutic correctors that were previously received and/or determined during a previous iteration of calculating inference model 208. Adherence training set 216 may be received by one or more remote devices that at least correlate a monitoring element and/or therapeutic corrector to an inference model, wherein a remote device is an external device to processor 104, as described above. Adherence training set may be received in the form of one or more user-entered correlations of a monitoring element and/or therapeutic corrector to an inference model.

Still referring to FIG. 2, processor 104 may receive adherence machine-learning model 212 from a remote device that utilizes one or more adherence machine learning processes, wherein a remote device is described above in detail. For example, and without limitation, a remote device may include a computing device, external device, processor, and the like thereof. Remote device may perform the adherence machine-learning process using the adherence training set to generate inference model 208 and transmit the output to processor 104. Remote device may transmit a signal, bit, datum, or parameter to processor 104 that at least relates to inference model 208. Additionally or alternatively, the remote device may provide an updated machine-learning model. For example, and without limitation, an updated machine-learning model may be comprised of a firmware update, a software update, an adherence machine-learning process correction, and the like thereof. As a non-limiting example a software update may incorporate a new monitoring element that relates to a modified therapeutic corrector. Additionally or alternatively, the updated machine learning model may be transmitted to the remote device, wherein the remote device may replace the adherence machine-learning model with the updated machine-learning model and calculate the inference model as a function of the monitoring using the updated machine-learning model. The updated machine-learning model may be transmitted by the remote device and received by processor 104 as a software update, firmware update, or corrected adherence machine-learning model. For example, and without limitation adherence machine-learning model may utilize a random forest machine-learning process, wherein the updated machine-learning model may incorporate a gradient boosting machine-learning process. Updated machine learning model may additionally or alternatively include any machine-learning model used as an updated machine learning model as described in U.S. Nonprovisional application Ser. No. 17/106,658, filed on Nov. 30, 2020, and entitled “A SYSTEM AND METHOD FOR GENERATING A DYNAMIC WEIGHTED COMBINATION,” the entirety of which is incorporated herein by reference.

Still referring to FIG. 2, processor 104 may calculate inference model 208 by generating a physiological progression parameter. As used in this disclosure a “physiological progression parameter” is a parameter associated with the overall timeline of a user's symptoms. For example, a user's symptoms may include a common cold, wherein a user may be on day 4 of the common cold, wherein it takes 8 days for the common cold to be eliminated. Additionally or alternatively, physiological progression parameter may include determining a percentage of elimination of a user's symptoms, wherein the percentage denotes the total amount of time with the symptoms and the total amount of time remaining. As a non-limiting example, a percentage of 22% may be identified for a user that is 22% completed with the symptoms and/or ailments. Physiological progression parameter may include determining the pace at which the symptoms and/or ailments are progressing. For example, and without limitation, physiological progression parameter may identify that a user's symptoms are progressing slowly and/or rapidly. Physiological progression parameter may be generated by identifying a status of therapeutic corrector 136. As user in this disclosure a “status” is a current state of a user's symptoms and/or therapeutic corrector 136 at a time period, wherein a time period includes seconds, minutes, hours, days, weeks, months, years, and the like thereof. Processor 104 may determine functional checkpoints as a function of therapeutic corrector 136. As used in this disclosure a “functional checkpoint” is a barrier and/or marker that is identified in therapeutic corrector 136 that represents a significant point in the physiologic process. As a non-limiting example, functional checkpoint may include a significant point associated with eliminating a cough and/or sneeze as a function of therapeutic corrector 136. As a further non-limiting example, functional checkpoint may include a significant point associated with a particular quantity and/or concentration of a biomarker in a user's body, wherein a “biomarker,” as used herein, is a molecule and/or chemical that identifies the health status of a user's body. Processor 104 generates physiological progression parameter as a function of functional checkpoint to at least determine the location and or progress of a user's symptoms in relation to the ailment.

Still referring to FIG. 2, processor 104 updates expert database 120 as a function of inference model 208 and therapeutic corrector 136. Updating expert database 120 may be conducted similarly to FIG. 1. Processor 104 may database 120 by generating a therapeutic framework. As used in this disclosure a “therapeutic framework” is a framework of programs, compilers, code, libraries, application programming interfaces, and/or data associated with therapeutic correctors and/or inference models. As a non-limiting example, therapeutic framework may include one or more decision support systems, web frameworks, cactus frameworks, and the like thereof. Information in therapeutic frame representing the generated therapeutic corrector and/or the calculated inference model may be collected, stored, and distributed. As a non-limiting example, therapeutic framework may be accessed by one or more advisors in one or more geographical locations. As a further non-limiting example, therapeutic framework may collect and store a plurality of inference models for a therapeutic corrector and/or a plurality of therapeutic correctors for an inference model. Therapeutic framework may be generated as a function of producing a cryptographic identifier. As used in this disclosure a “cryptographic identifier” is data that is obtained as user identifier data that is cryptographically altered and/or secured. In an embodiment and without limitation, cryptographic identifier prevents user identifier from being accessed on therapeutic framework, such that only therapeutic corrector and/or inference models are presented on therapeutic framework.

Still referring to FIG. 2, cryptographic identifier may include data that has been converted by a cryptographic system. In one embodiment, a cryptographic system is a system that converts data from a first form, known as “plaintext,” which is intelligible when viewed in its intended format, into a second form, known as “ciphertext,” which is not intelligible when viewed in the same way. Ciphertext may be unintelligible in any format unless first converted back to plaintext. In one embodiment, a process of converting plaintext into ciphertext is known as “encryption.” Encryption process may involve the use of a datum, known as an “encryption key,” to alter plaintext. Cryptographic system may also convert ciphertext back into plaintext, which is a process known as “decryption.” Decryption process may involve the use of a datum, known as a “decryption key,” to return the ciphertext to its original plaintext form. In embodiments of cryptographic systems that are “symmetric,” decryption key is essentially the same as encryption key: possession of either key makes it possible to deduce the other key quickly without further secret knowledge. Encryption and decryption keys in symmetric cryptographic systems may be kept secret and shared only with persons or entities that the user of the cryptographic system wishes to be able to decrypt the ciphertext. One example of a symmetric cryptographic system is the Advanced Encryption Standard (“AES”), which arranges plaintext into matrices and then modifies the matrices through repeated permutations and arithmetic operations with an encryption key.

In embodiments of cryptographic systems that are “asymmetric,” either encryption or decryption key cannot be readily deduced without additional secret knowledge, even given the possession of a corresponding decryption or encryption key, respectively; a common example is a “public key cryptographic system,” in which possession of the encryption key does not make it practically feasible to deduce the decryption key, so that the encryption key may safely be made available to the public. An example of a public key cryptographic system is RSA, in which an encryption key involves the use of numbers that are products of very large prime numbers, but a decryption key involves the use of those very large prime numbers, such that deducing the decryption key from the encryption key requires the practically infeasible task of computing the prime factors of a number which is the product of two very large prime numbers. Another example is elliptic curve cryptography, which relies on the fact that given two points P and Q on an elliptic curve over a finite field, and a definition for addition where A+B=R, the point where a line connecting point A and point B intersects the elliptic curve, where “0,” the identity, is a point at infinity in a projective plane containing the elliptic curve, finding a number k such that adding P to itself k times results in Q is computationally impractical, given correctly selected elliptic curve, finite field, and P and Q.

Processor 104 is configured to receive an advisory input (e.g., first advisory input 112) including a constitutional inquiry. The advisory input and the constitutional inquiry is further described in detail in this disclosure.

With continued reference to FIG. 2, processor 104 is configured to generate, using a large language model (LLM) 220 and one or more machine-learning modules 224a-n, a first therapeutic corrector 226a as a function of the advisory input, wherein the LLM 220 has been trained with first LLM training data 228 including correlations between keywords. For the purposes of this disclosure, a “machine-learning module” is a hardware and/or software component configured to execute at least one machine-learning process that produces an output from an input. In some cases, machine-learning module 224a-n may include supervised learning algorithms, unsupervised learning algorithms, semi-supervised learning algorithms, reinforcement learning algorithms, or hybrid learning algorithms. As a non-limiting example, machine-learning modules 224a-n may include LLM 220, first machine-learning module 224a, second machine-learning module 224b, third machine-learning module 224c, scoring machine-learning model 224d, web crawling module 224e, constitutional generator module 108, constitutional advisory module 140, best practices module 148, language processing module 124, and the like as described in this disclosure. As a non-limiting example, first therapeutic corrector 226a may include one or more proposed therapeutic actions, such as a recommended diagnosis, treatment, laboratory test, medical procedure, or other remedial or evaluative measure relevant to constitutional inquiry. In some embodiments, first therapeutic corrector 226a may include ranked or prioritized options, confidence scores, or explanatory text indicating the rationale for the proposed actions. In some cases, first therapeutic corrector 226a may be displayed on an advisor client device 132 using a graphical user interface (GUI) 128.

Still referring to FIG. 2, a “large language model,” as used herein, is a deep learning data structure that can recognize, summarize, translate, predict and/or generate text and other content based on knowledge gained from massive datasets. Large language models 220 may be trained on large sets of data. In some embodiments, training sets of an LLM 220 may include information from one or more public or private databases. In an embodiment, an LLM 220 may include one or more architectures based on capability requirements of an LLM 220. Exemplary architectures may include, without limitation, GPT (Generative Pretrained Transformer), BERT (Bidirectional Encoder Representations from Transformers), T5 (Text-To-Text Transfer Transformer), and the like. Architecture choice may depend on a needed capability such generative, contextual, or other specific capabilities.

With continued reference to FIG. 2, in some embodiments, an LLM 220 may include and/or be produced using Generative Pretrained Transformer (GPT), GPT-2, GPT-3, GPT-4, and the like. GPT, GPT-2, GPT-3, GPT-3.5, and GPT-4 are products of Open AI Inc., of San Francisco, CA. An LLM 220 may include a text prediction based algorithm configured to receive an article and apply a probability distribution to the words already typed in a sentence to work out the most likely word to come next in augmented articles. For example, if some words that have already been typed are “Nice to meet,” then it may be highly likely that the word “you” will come next. An LLM 220 may output such predictions by ranking words by likelihood or a prompt parameter. For the example given above, an LLM 220 may score “you” as the most likely, “your” as the next most likely, “his” or “her” next, and the like. An LLM 220 may include an encoder component and a decoder component.

Still referring to FIG. 2, LLM 220 may include an attention mechanism, utilizing a transformer as described further below. An “attention mechanism,” as used herein, is a part of a neural architecture that enables a system to dynamically highlight relevant features of the input data. In natural language processing this may be a sequence of textual elements. It may be applied directly to the raw input or to its higher-level representation. An attention mechanism may be an improvement to the limitation of the Encoder-Decoder model which encodes the input sequence to one fixed length vector from which to decode the output at each time step. This issue may be seen as a problem when decoding long sequences because it may make it difficult for the neural network to cope with long sentences, such as those that are longer than the sentences in the training corpus. Applying an attention mechanism, LLM 220 may predict the next word by searching for a set of position in a source sentence where the most relevant information is concentrated. LLM 220 may then predict the next word based on context vectors associated with these source positions and all the previous generated target words, such as textual data of a dictionary correlated to a prompt in a training data set. A “context vector,” as used herein, are fixed-length vector representations useful for document retrieval and word sense disambiguation. In some embodiments, LLM 220 may include encoder-decoder model incorporating an attention mechanism.

Still referring to FIG. 2, LLM 220 may include a transformer architecture. In some embodiments, encoder component of LLM 220 may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. “Positional encoding,” for the purposes of this disclosure, refers to a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 2, attention mechanism may represent an improvement over a limitation of an encoder-decoder model. An encoder-decider model encodes an input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, an LLM 220 may predict the next word by searching for a set of positions in a source sentence where the most relevant information is concentrated. An LLM 220 may then predict the next word based on context vectors associated with these source positions and all the previously generated target words, such as textual data of a dictionary correlated to a prompt in a training data set. A “context vector,” as used herein, are fixed-length vector representations useful for document retrieval and word sense disambiguation.

Still referring to FIG. 2, attention mechanism may include, without limitation, generalized attention self-attention, multi-head attention, additive attention, global attention, and the like. In generalized attention, when a sequence of words or an image is fed to an LLM 220, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, an LLM 220 may pick up particular parts at different positions in the input sequence and over time compute an initial composition of the output sequence. In multi-head attention, an LLM 220 may include a transformer model of an attention mechanism. Attention mechanisms, as described above, may provide context for any position in the input sequence. For example, if the input data is a natural language sentence, the transformer does not have to process one word at a time. In multi-head attention, computations by an LLM 220 may be repeated over several iterations, each computation may form parallel layers known as attention heads. Each separate head may independently pass the input sequence and corresponding output sequence element through a separate head. A final attention score may be produced by combining attention scores at each head so that every nuance of the input sequence is taken into consideration. In additive attention (Bahdanau attention mechanism), an LLM 220 may make use of attention alignment scores based on a number of factors. Alignment scores may be calculated at different points in a neural network, and/or at different stages represented by discrete neural networks. Source or input sequence words are correlated with target or output sequence words but not to an exact degree. This correlation may take into account all hidden states and the final alignment score is the summation of the matrix of alignment scores. In global attention (Luong mechanism), in situations where neural machine translations are required, an LLM 220 may either attend to all source words or predict the target sentence, thereby attending to a smaller subset of words.

With continued reference to FIG. 2, multi-headed attention in encoder may apply a specific attention mechanism called self-attention. Self-attention allows models such as an LLM 220 or components thereof to associate each word in the input to other words. As a non-limiting example, an LLM 220 may learn to associate the word “you,” with “how” and “are.” It is possible that an LLM 220 learns that words structured in this pattern are typically a question and to respond appropriately. In some embodiments, to achieve self-attention, input may be fed into three distinct fully connected neural network layers to create query, key, and value vectors. A query vector may include an entity's learned representation for comparison to determine attention score. A key vector may include an entity's learned representation for determining the entity's relevance and attention weight. A value vector may include data used to generate output representations. Query, key, and value vectors may be fed through a linear layer; then, the query and key vectors may be multiplied using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

Still referencing FIG. 2, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power.

With continued reference to FIG. 2, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

Continuing to refer to FIG. 2, transformer architecture may include a decoder. Decoder may be a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input.

With further reference to FIG. 2, in some embodiments, input to decoder may go through an embedding layer and positional encoding layer in order to obtain positional embeddings. Decoder may include a first multi-headed attention layer, wherein the first multi-headed attention layer may receive positional embeddings.

With continued reference to FIG. 2, first multi-headed attention layer may be configured to not condition to future tokens. As a non-limiting example, when computing attention scores on the word “am,” decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “0s” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens.”

Still referring to FIG. 2, second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 2, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word.

Still referring to FIG. 2, decoder may take this output and add it to the decoder inputs. Decoder may continue decoding until a token is predicted. Decoder may stop decoding once it predicts an end token.

Continuing to refer to FIG. 2, in some embodiment, decoder may be stacked N layers high, with each layer taking in inputs from the encoder and layers before it. Stacking layers may allow an LLM 220 to learn to extract and focus on different combinations of attention from its attention heads.

With continued reference to FIG. 2, in some embodiments, processor 104 may incorporate retrieval augmented generation (RAG) into LLM 220. For the purposes of this disclosure, “retrieval-augmented generation” is a method that enhances a response generation capability of a large language model by integrating external, relevant information retrieved from a structured database or unstructured corpus. In some embodiments, by leveraging RAG, LLM 220 can reduce a risk of generating incorrect or hallucinated information, instead relying on curated and contextually relevant data. For the purposes of this disclosure, “hallucination” of information refers to where a language model fabricates plausible-sounding but incorrect information. In some embodiments, processor 104 may retrieve relevant information as a function element from internal or external medical database and the retrieved data may be input into LLM 220 to generate responses (therapeutic corrector 136) grounded in authoritative sources. In some embodiments, processor 104 may identify keywords or semantic elements in the query and using these elements to search a database for information.

With continued reference to FIG. 2, in some embodiments, processor 104 may utilize similarity-based fetching techniques to identify most relevant data for input to LLM 220. For the purposes of this disclosure, “similarity-based fetching” is a process by which a query is converted into a high-dimensional vector embedding, representing its semantic meaning, and compared with pre-computed embeddings of documents or data in a database. In some embodiments, retrieved documents with high similarity scores may be integrated into an input for LLM 220. In some embodiments, processor 104 may select an appropriate database for a given query based on context and sensitivity of information. For instance, and without limitation, queries containing identifiable patient information may restrict retrieval to private internal sources. In some embodiments, LLM 220 may generate an initial response based on an input query, and this response may be then analyzed to identify additional relevant keywords or concepts. In some embodiments, these elements may subsequently be used to perform a second round of data retrieval. In a non-limiting example, additional retrieved data may then be input into LLM 220 alongside the original query and first response to generate an output (e.g., therapeutic corrector 136).

With continued reference to FIG. 2, in some embodiments, processor 104 may generate hypothetical document embeddings. For the purposes of this disclosure, a “hypothetical document embedding” refers to an embedding created by LLM that represents its semantic understanding of a query or preliminary response. In some embodiments, the embeddings may be compared against database embeddings to identify documents or data closely aligned with the system's understanding of a query. In some embodiments, the retrieved information may then be incorporated into an input of LLM 220.

With continued reference to FIG. 2, an LLM 220 may receive an input. Input may include a string of one or more characters. Inputs may additionally include unstructured data. For example, input may include one or more words, a sentence, a paragraph, a thought, a query, and the like. A “query” for the purposes of the disclosure is a string of characters that poses a question. In some embodiments, input may be received from a user device. User device may be any computing device that is used by a user. As non-limiting examples, user device may include desktops, laptops, smartphones, tablets, and the like. In some embodiments, input may include any set of data associated with feedback input 232, advisory input, expert input 116, adherence input 204, and the like.

With continued reference to FIG. 2, an LLM 220 may generate therapeutic corrector 136 (first therapeutic corrector 226a, second therapeutic corrector 226b, and the like) as an output. In some cases, first therapeutic corrector 226a and second therapeutic corrector 226b may be consistent with therapeutic corrector 136. In some embodiments, an LLM 220 may include multiple sets of transformer architecture as described above. Output may include a textual output. A “textual output,” for the purposes of this disclosure is an output comprising a string of one or more characters. In some embodiments, textual output may include a phrase or sentence identifying the status of first advisory input. In some embodiments, textual output may include a sentence or plurality of sentences describing a response to a first advisory input.

With continued reference to FIG. 2, for the purposes of this disclosure, “first LLM training data” is a collection of data elements including correlations between keywords, wherein the correlations represent statistical, semantic, or contextual relationships derived from one or more corpora. In some cases, first LLM training data 228 may include, without limitation, tokens, n-grams, keywords, or phrases associated with one another based on co-occurrence frequencies, syntactic dependencies, semantic similarity scores, or domain-specific knowledge representations. In some embodiments, first LLM training data 228 may be obtained from structured data sources, unstructured text, or multimodal datasets. In some embodiments, first LLM training data 228 may include domain-agnostic corpora and/or domain-specific corpora. In some embodiments, first LLM training data 228 may be used to initialize, pretrain, or fine-tune a large language model 220 to generate first therapeutic correctors 226a, in response to advisory inputs.

With continued reference to FIG. 2, in some embodiments, first LLM training data 228 may include general training sets 236. In some cases, LLM 220 may have been generally trained with general training sets 236 of first LLM training data 228, wherein the general training sets 236 may include correlations between linguistic terms associated with a particular data domain 240. For the purposes of this disclosure, “general training sets” are a subset of first LLM training data including correlations between linguistic terms to generally train a large language model. As a non-limiting example, general training sets 236 may include large-scale text datasets such as encyclopedic content, news articles, technical manuals, literature, academic publications, conversational transcripts, and other publicly or privately available sources. In some cases, general training sets 236 may capture statistical, semantic, and syntactic relationships (e.g., tone, style, and the like) between words, phrases, and concepts across a wide variety of contexts. In some embodiments, general training sets 236 may be preprocessed to remove low-quality, redundant, or irrelevant content, normalized to a standard tokenization format, and annotated with metadata such as source type, publication date, or language variant. The linguistic terms and data domain 240 are described in detail below.

With continued reference to FIG. 2, in some cases, first LLM training data 228 may include specific training sets 244. In some cases, LLM 220 may have been specifically trained with specific training sets 244 of the first LLM training data 228, wherein the specific training sets 244 may include exemplary advisory inputs correlated to exemplary therapeutic correctors. For the purposes of this disclosure, “specific training sets” are a subset of first LLM training data including curated correlations between advisory inputs and therapeutic correctors. As a non-limiting example, specific training sets 244 may include exemplary advisory inputs paired with validated therapeutic correctors, domain-specific vocabularies, procedural guidelines, expert-authored reference materials, structured medical or technical databases, regulatory documentation, and historical case records.

With continued reference to FIG. 2, in some embodiments, an LLM 220 may be generally trained. As used in this disclosure, a “generally trained” LLM is an LLM that is trained on a general training set comprising a variety of subject matters, data sets, and fields. In some embodiments, an LLM 220 may be initially generally trained. Additionally, or alternatively, an LLM 220 may be specifically trained. As used in this disclosure, a “specifically trained” LLM is an LLM that is trained on a specific training set, wherein the specific training set includes data including specific correlations for the LLM to learn. As a non-limiting example, an LLM 220 may be generally trained on a general training set 236, then specifically trained on a specific training set 244. In an embodiment, specific training of an LLM 220 may be performed using a supervised machine learning process. In some embodiments, generally training an LLM 220 may be performed using an unsupervised machine learning process. As a non-limiting example, specific training set 244 may include information from a database. As a non-limiting example, specific training set may include text related to users correlated to examples of outputs. In an embodiment, training one or more machine learning models may include setting the parameters of the one or more models (weights and biases) either randomly or using a pretrained model. Generally training one or more machine learning models on a large corpus of text data can provide a starting point for fine-tuning on a specific task. A model such as an LLM 220 may learn by adjusting its parameters during the training process to minimize a defined loss function, which measures the difference between predicted outputs and ground truth. Once a model has been generally trained, the model may then be specifically trained to fine-tune the pretrained model on task-specific data to adapt it to the target task. Fine-tuning may involve training a model with specific training set 244, adjusting the model's weights to optimize performance for the particular task. In some cases, this may include optimizing the model's performance by fine-tuning hyperparameters such as learning rate, batch size, and regularization. Hyperparameter tuning may help in achieving the best performance and convergence during training. In an embodiment, fine-tuning a pretrained model such as an LLM 220 may include fine-tuning the pretrained model using Low-Rank Adaptation (LoRA). As used in this disclosure, “Low-Rank Adaptation” is a training technique for large language models that modifies a subset of parameters in the model. Low-Rank Adaptation may be configured to make the training process more computationally efficient by avoiding a need to train an entire model from scratch. In an exemplary embodiment, a subset of parameters that are updated may include parameters that are associated with a specific task or domain.

With continued reference to FIG. 2, in some embodiments, LLM 220 may be specifically trained using specific training sets 244. In some embodiments, specific training sets 244 may include a set of data that is in user's voice, email, or the like to mimic them. In some embodiments, specific training sets 244 may be consistent with any training data described in the entirety of this disclosure. In some embodiments, specific training sets 244 may be received from one or more users, database, external computing devices, and/or previous iterations of processing. As a non-limiting example, specific training sets 244 may include instructions from a user, who may be an expert user, a past user in embodiments disclosed herein, or the like, which may be stored in memory and/or stored in database, where the instructions may include labeling of training examples. In some embodiments, specific training sets 244 may be updated iteratively through a feedback loop. In some embodiments, processor 104 may be configured to generate LLM 220. In a non-limiting example, generating LLM 220 may include training, retraining, or fine-tuning LLM 220 using specific training sets 244 or updated specific training sets 244.

With continued reference to FIG. 2, in some cases, generating first therapeutic corrector 226a may include determining at least a treatment using a first machine-learning module 224a of one or more machine-learning modules 224a-n that has been trained with first training data including exemplary advisory inputs correlated to exemplary treatments. For the purposes of this disclosure, a “treatment” is a therapeutic intervention, regimen, or course of action intended to prevent, mitigate, or cure a medical condition or to otherwise improve a patient's health status. As a non-limiting example, treatment may include administration of pharmaceutical drugs, surgical procedures, physical therapy, lifestyle modifications, dietary interventions, or combinations thereof. In some embodiments, a treatment may be specified in terms of dosage, route of administration, duration, and timing. In some embodiments, a treatment may be selected based on patient-specific factors such as age, comorbidities, allergies, or genetic profile. In a non-limiting example, processor 104 may identify that advisory input requires a treatment-related output, either because the advisory input explicitly contains terms or phrases indicating a request for a treatment recommendation, or because a preliminary analysis by LLM 220 has classified the advisory input into a treatment-oriented category. The processor 104 may then route the advisory input, or a preprocessed representation thereof, to first machine-learning module 224a. The first training data used for the first machine-learning module 224a may include a plurality of labeled examples, each including an advisory input (such as a patient's symptoms, medical history, demographic information, and contextual notes) paired with an exemplary treatment (such as a specific drug and dosage, a surgical procedure, a physical therapy plan, or a combination of interventions). In some cases, second machine-learning module 224b may apply one or more statistical or neural network-based inference methods, such as multi-class classification, probabilistic reasoning, or ensemble models, to produce one or more candidate diagnoses. Feature extraction methods may be applied to advisory input, including tokenization, embedding generation, and the extraction of structured data features from free-text narratives.

With continued reference to FIG. 2, in some cases, generating first therapeutic corrector 226a may include determining at least a diagnosis using a second machine-learning module 224b of one or more machine-learning modules 224a-n that has been trained with second training data including exemplary advisory inputs correlated to exemplary diagnoses. For the purposes of this disclosure, a “diagnosis” is an identification or classification of a disease, disorder, condition, or health status of a patient. In some cases, diagnosis may include classification code (e.g., ICD code), a descriptive medical term, or bot. In some cases, diagnosis may include primary diagnoses as well as differential diagnoses indicating possible alternative conditions. In a non-limiting example, processor 104 may determine that advisory input involves a diagnostic determination. This identification may occur because the advisory input explicitly seeks a diagnosis (e.g., “What condition matches these symptoms?”) or because a classification step performed by LLM 220 or another triage model has labeled the input as diagnostic in nature. Once classified, the processor 104 may route the advisory input, or a preprocessed representation thereof, to second machine-learning module 224b. In some cases, second training data used for this module may include a plurality of labeled examples, each including an advisory input (such as symptom descriptions, medical history, demographic details, imaging results, laboratory test summaries, or clinical notes) paired with an exemplary diagnosis. In some cases, second machine-learning module 224b may apply one or more statistical or neural network-based inference methods, such as multi-class classification, probabilistic reasoning, or ensemble models, to produce one or more candidate diagnoses ranked by likelihood scores. Feature extraction methods may be applied to the advisory input, including tokenization, embedding generation, and the extraction of structured data features from free-text narratives.

With continued reference to FIG. 2, in some cases, generating first therapeutic corrector 226a may include determining at least a lab work using a third machine-learning module 224c of one or more machine-learning modules 224a-n that has been trained with third training data including exemplary advisory inputs correlated to exemplary lab works. For the purposes of this disclosure, a “lab work” is a clinical test or set of tests performed on biological samples. As a non-limiting example, lab work may include complete blood count (CBC), metabolic panels, microbiological cultures, polymerase chain reaction (PCR) assays, imaging-assisted biopsies, or genetic sequencing. In some embodiments, lab work may be recommended to confirm a diagnosis, guide treatment selection, monitor treatment effectiveness, or assess disease progression. In a non-limiting example, processor 104 may determine that advisory input is associated with or inquiring recommendation of laboratory testing or other diagnostic procedures involving biological sample analysis. This determination may occur because the advisory input explicitly requests information about tests to perform (e.g., “What lab work should be ordered for these symptoms?”) or because a classification function within LLM 220 or a separate rule-based or statistical model has flagged the input as requiring lab work recommendations. The advisory input, or its preprocessed form, may then be routed to third machine-learning module 224c. In some cases, third training data may include a plurality of labeled examples, each pairing an advisory input, such as patient symptoms, medical history, demographic data, provisional diagnoses, or physical examination findings, with an exemplary lab work recommendation. As a non-limiting example, lab works may include standard clinical laboratory tests (e.g., complete blood count, comprehensive metabolic panel, urinalysis), specialized diagnostic assays (e.g., polymerase chain reaction [PCR] for infectious disease detection, tumor marker analysis, genetic sequencing), or procedural investigations involving laboratory analysis of specimens (e.g., biopsy with histopathology). In some cases, third machine-learning module 224c may extract structured and unstructured features from the advisory input, apply embedding or vectorization techniques to represent the input in a machine-readable form, and process it through a predictive model such as a classification network, ranking algorithm, or hybrid probabilistic model.

With continued reference to FIG. 2, processor 104 is configured to receive a feedback input 232 in response to an implementation of first therapeutic corrector 226a, wherein at least a part of the feedback input 232 indicates that the first therapeutic corrector 226a is incorrect. For the purposes of this disclosure, a “feedback input” is any data, signal, or communication that describes, evaluates, or otherwise characterizes results of implementing a therapeutic corrector. As a non-limiting example, feedback input 232 may include qualitative information, such as narrative descriptions of clinical observations, user satisfaction assessments, or textual commentary. As another non-limiting example, feedback input 232 may include quantitative information, such as numerical outcome measures, biomarker readings, or performance metrics. In some embodiments, feedback input 232 may explicitly indicate that a first therapeutic corrector 226a is correct, partially correct, or incorrect. In other embodiments, feedback input 232 may include structured fields such as checklists, rating scales, or standardized scoring forms. In some cases, feedback input 232 may be provided through a graphical user interface 128, an application programming interface, or a connected device. In some cases, feedback input 232 may include time stamps, contextual metadata, or identifiers associating feedback input 232 with a particular advisory input, therapeutic corrector, or user. As a non-limiting example, feedback input 232 may include a statement, selection, measurement, or other data element that indicates that first therapeutic corrector 226a, or a portion thereof, is incorrect. As a non-limiting example, an informed advisor may provide a textual description stating that a recommended treatment contained in the first therapeutic corrector was clinically inappropriate for the user's condition and may select a “not accurate” or “not applicable” option from a feedback interface. In some cases, feedback input 232 may indicate that first therapeutic corrector 226a, or a portion thereof, is correct. As a non-limiting example, an informed advisor may submit a narrative report stating that the recommended treatment contained in the first therapeutic corrector was implemented and resulted in the expected clinical improvement and may select a “confirmed accurate” or “appropriate” option from a feedback interface.

With continued reference to FIG. 2, in some embodiments, processor 104 may be configured to generate a user interface displaying therapeutic corrector 136, first therapeutic corrector 226a, second therapeutic corrector 226b, and the like. For the purposes of this disclosure, a “user interface” is a means by which a user and a computer system interact. For example through the use of input devices and software. A user interface may include a graphical user interface (GUI) 128, command line interface (CLI), menu-driven user interface, touch user interface, voice user interface (VUI), form-based user interface, any combination thereof and the like. In some embodiments, user interface may operate on and/or be communicatively connected to a decentralized platform, metaverse, and/or a decentralized exchange platform associated with a user. For example, a user may interact with user interface in virtual reality. In some embodiments, a user may interact with the user interface using a computing device distinct from and communicatively connected to at least a processor 104. For example, a smart phone, smart, tablet, or laptop operated by a user. In an embodiment, user interface may include a graphical user interface 128. A “graphical user interface,” as used herein, is a graphical form of user interface that allows users to interact with electronic devices. In some embodiments, GUI 128 may include icons, menus, other visual indicators or representations (graphics), audio indicators such as primary notation, and display information and related user controls. A menu may contain a list of choices and may allow users to select one from them. A menu bar may be displayed horizontally across the screen such as pull-down menu. When any option is clicked in this menu, then the pull-down menu may appear. A menu may include a context menu that appears only when the user performs a specific action. An example of this is pressing the right mouse button. When this is done, a menu may appear under the cursor. Files, programs, web pages and the like may be represented using a small picture in a graphical user interface. For example, links to decentralized platforms as described in this disclosure may be incorporated using icons. Using an icon may be a fast way to open documents, run programs etc. because clicking on them yields instant access.

With continued reference to FIG. 2, processor 104 is configured to generate a feedback quality score 248 as a function of feedback input 232 using a scoring machine-learning model 224d of one or more machine-learning modules 224a-n that has been trained with scoring training data including exemplary feedback inputs correlated to exemplary feedback quality scores. For the purposes of this disclosure, a “feedback quality score” is a quantitative or qualitative metric to represent an evaluation of the reliability, accuracy, effectiveness, and/or usefulness of a therapeutic corrector. In some embodiments, feedback quality score 248 may be expressed as a numerical value, such as a percentage or a point score. In some embodiments, feedback quality score 248 may be expressed as a qualitative classification, such as “high,” “medium,” or “low” quality.

With continued reference to FIG. 2, for the purposes of this disclosure, a “scoring machine-learning model” is a hardware and/or software component configured to execute one or more machine-learning processes for generating a feedback quality score from a received feedback input. In some cases, scoring machine-learning model 224d may be implemented using supervised learning algorithms, unsupervised learning algorithms, reinforcement learning algorithms, or combinations thereof. In some cases, scoring machine-learning model 224d may be trained to recognize patterns, features, or correlations within feedback inputs 232 that are indicative of accuracy, reliability, completeness, or clinical utility. In some embodiments, scoring machine-learning model may include a feature extraction engine for parsing textual, numerical, or multimodal elements of a feedback input, a model parameter optimizer for adjusting internal weights based on training data and an output layer configured to produce feedback quality score 248 in a numerical, categorical, or probabilistic form. In some cases, scoring machine-learning model 224d may be consistent with any machine-learning model described in this disclosure. For the purposes of this disclosure, “scoring training data” is a dataset used to train a scoring machine-learning model. In some cases, scoring training data may be consistent with any training data described in this disclosure. In some embodiments, processor 104 may be configured to generate scoring training data. In a non-limiting example, scoring training data may include correlations between exemplary feedback input, exemplary inference input and exemplary feedback quality scores. In some embodiments, scoring training data may be stored in database. In some embodiments, scoring training data may be received from one or more users, database, external computing devices, and/or previous iterations of processing. As a non-limiting example, scoring training data may include instructions from a user, who may be an expert user, a past user in embodiments disclosed herein, or the like, which may be stored in memory and/or stored in database, where the instructions may include labeling of training examples. In some embodiments, scoring training data may be updated iteratively on a feedback loop. As a non-limiting example, processor 104 may update scoring training data iteratively through a feedback loop as a function of first advisory input 112, adherence input 204, feedback input 232, data cohort, or the like. In some embodiments, processor 104 may be configured to generate a scoring machine-learning model 224d. In a non-limiting example, generating scoring machine-learning model 224d may include training, retraining, or fine-tuning scoring machine-learning model 224d using scoring training data or updated scoring training data. In some embodiments, scoring machine-learning model 224d may have been trained with scoring training data. In some embodiments, processor 104 may be configured to determine feedback quality score 248 using scoring machine-learning model 224d (i.e. trained or updated scoring machine-learning model 224d). In some embodiments, scoring machine-learning model 224d may receive feedback input 232 as inputs and may output feedback quality score 248 in response to the inputs. In some embodiments, scoring machine-learning model 224d may function differently between training time and inference time. In a non-limiting example, at training time, processor 104 may be configured to train, retrain, or fine-tune scoring machine-learning model 224d using scoring training data. During the training time, scoring machine-learning model 224d may learn to associate patterns within feedback input 232. In a non-limiting example, at inference time, trained scoring machine-learning model 224d may be configured to receive previously unseen feedback input 232 and, based on the representations learned during training time, automatically output a selection of feedback quality score 248. Inference may be triggered in response to a user request, system event, or automated workflow operation. In some cases, user may manually input feedback quality score 248.

With continued reference to FIG. 2, in some cases, generating feedback quality score 248 may include receiving an adherence input 204 from a monitoring device and generating the feedback quality score 248 as a function of feedback input 232 and the adherence input 204.

In some cases, processor 104 may be configured to interface with one or more monitoring devices, which may include wearable health trackers, implantable medical sensors, home medical equipment, or remote patient monitoring systems. These devices may continuously or periodically collect adherence input 204, such as timestamps of medication intake, dosage compliance logs, biometric readings indicating whether a prescribed activity or treatment was followed (e.g., heart rate patterns during prescribed exercise), or environmental measurements relevant to treatment adherence (e.g., CPAP machine usage logs for sleep apnea therapy). In some cases, processor 104 may receive adherence input 204 in raw form, such as sensor data streams or time-stamped event logs, and may preprocess it by normalizing the values, filtering noise, and aligning it with the relevant timeframes for which first therapeutic corrector 226a was in effect. The processor 104 may then combine this adherence input 204 with feedback input 232 such as qualitative assessments, numerical ratings, or narrative reports from an informed advisor or end-user. The combination may be performed by a scoring machine-learning model 224d, which may be trained to treat adherence input 204 as either a weighted feature in a regression or classification framework or as a separate factor in a multi-objective optimization process. For example, and without limitation, a treatment recommendation that received positive qualitative feedback but showed low adherence rates may result in a lower feedback quality score, indicating that despite favorable perception, the treatment was impractical or burdensome to follow. For example, and without limitation, high adherence data corroborating positive outcome feedback may yield a higher feedback quality score, reflecting both acceptability and effectiveness.

With continued reference to FIG. 2, processor 104 is configured to update LLM 220 and one or more machine-learning modules 224a-n as a function of at least in part on feedback quality score 248, wherein updating includes generating second LLM training data 252 including first LLM training data 228 and therapeutic correctors that are incorrectly generated using the first LLM training data 228 and generating a second therapeutic corrector 226b using the updated LLM 220 that is retrained with the second LLM training data 252. Processor 104 is configured to generate a graphical user interface 128 displaying second therapeutic corrector 226b. For the purposes of this disclosure, “second LLM training data” is a collection of data elements generated or selected after evaluation of a first therapeutic corrector. In some cases, second LLM training data may include first LLM training data 228 augmented with additional data entries corresponding to therapeutic correctors that were determined to be incorrect, partially correct, or suboptimal based on one or more feedback quality scores 248. In a non-limiting example, if feedback quality score 248 indicates that a recommendation in first therapeutic corrector 226a was incorrect (e.g., it proposed an inappropriate treatment), partially correct (e.g., it included some valid elements but omitted or misstated others), or suboptimal (e.g., it was less effective than alternative options), processor 104 may flag that the first therapeutic corrector 226a for inclusion in second LLM training data 252. In some cases, second LLM training data 252 may be consistent with first LLM training data 228. In some embodiments, second LLM training data 252 may be stored in database. In some embodiments, second LLM training data 252 may be received from one or more users, database, external computing devices, and/or previous iterations of processing. As a non-limiting example, second LLM training data 252 may include instructions from a user, who may be an expert user, a past user in embodiments disclosed herein, or the like, which may be stored in memory and/or stored in database, where the instructions may include labeling of training examples. In some embodiments, second LLM training data 252 may be updated iteratively on a feedback loop. As a non-limiting example, processor 104 may update second LLM training data 252 iteratively through a feedback loop as a function of feedback quality score 248, or the like.

With continued reference to FIG. 2, in some cases, updating LLM 220 may include modifying specific training sets 244 by generating a synthetic data pair 256 incorporating first advisory input and first therapeutic corrector 226a with feedback quality score 248 lower than a score threshold and augmenting the specific training sets 244 with the synthetic data pair 256, wherein augmenting the specific training sets 244 may include re-weighting one or more existing data pairs in the specific training sets 244 that include correlations related to correlations in the synthetic data pair 256 based on the feedback quality score 248. For the purposes of this disclosure, a “synthetic data pair” is an artificially generated association between an advisory input and a corresponding therapeutic corrector. In some cases, processor 104 may apply a generative model, such as LLM 220 or another text generation engine, to produce a synthetic data pair 256 in which advisory input is paired with a corrected or optimized therapeutic corrector that reflects the insights derived from feedback input 232 and feedback quality score. In a non-limiting example, synthetic data pair 256 may include an advisory input paired with a therapeutic corrector that has been revised to address deficiencies identified in the original output. In another non-limiting example, synthetic data pair 256 may include an advisory input paired with a therapeutic corrector that has been identified or indicated as incorrect. In some cases, processor 104 may first analyze synthetic data pair 256, which includes first advisory input and first therapeutic corrector 226a having a feedback quality score 248 lower than a score threshold, to extract one or more correlations between keywords, phrases, concepts, or other language features present in the synthetic data pair 256. In some cases, processor 104 may compare these extracted correlations to correlations present in the existing data pairs of specific training sets 244, identifying those existing pairs that contain matching or semantically similar correlations.

With continued reference to FIG. 2, for the purposes of this disclosure, a “score threshold” is a predefined numerical or categorical limit used to determine whether a feedback quality score associated with a therapeutic corrector meets or fails to meet a minimum standard of reliability, accuracy, effectiveness, or usefulness. In some cases, score threshold may be expressed as a fixed value. As a non-limiting example, score threshold may include 0.75 on a normalized 0 to 1 scale. In some cases, score threshold may be expressed as a categorical rating. As a non-limiting example, score threshold may include “medium” quality. In some cases, score threshold may be selected based on empirical testing, expert consensus, regulatory requirements, or performance criteria established for the large language model 220. In some embodiments, score threshold may be static and applied uniformly to all feedback quality scores 248. In some embodiments, score threshold may be dynamic and vary as a function of contextual factors, such as the domain of the advisory input, the criticality of the decision supported by the therapeutic corrector, or the level of confidence in the feedback input itself. In some cases, user may manually input score threshold. In a non-limiting example, a feedback quality score 248 lower than a score threshold may be interpreted as an indication that the corresponding therapeutic corrector 226a is incorrect, partially correct, or suboptimal, thereby triggering actions such as generating a synthetic data pair 256, re-weighting existing data pairs containing related correlations, or otherwise adjusting specific training sets 244 during model retraining. In another non-limiting example, a feedback quality score 248 equal to or greater than a score threshold may be treated as an indication of sufficient correctness or usefulness, potentially reinforcing related data correlations during training.

With continued reference to FIG. 2, in some cases, similarity between correlations may be determined using vector embeddings of terms or term pairs, cosine similarity metrics, or other semantic distance measures. In other cases, the comparison may be based on pattern matching between structured relationships, such as advisory input-therapeutic corrector mappings. Once related correlations are identified, processor 104 may adjust the weight values of the corresponding existing data pairs to reduce their influence on model retraining, with the magnitude of the adjustment being proportional to or otherwise determined by feedback quality score 248 of synthetic data pair 256. For example, and without limitation, a synthetic data pair 256 with a very low feedback quality score may trigger a more significant reduction in the weight of related existing data pairs, while a feedback quality score just below a score threshold may cause a smaller adjustment.

With continued reference to FIG. 1, in some cases, updating LLM 220 may include modifying general training sets 236 by selecting one data domain 240 from a plurality of data domains 240 as a function of feedback quality score 248 and modifying the general training sets 236 to include linguistic terms including the selected data domain 240. For the purposes of this disclosure, a “data domain” is a categorized subset of training data that pertain to a specific topical area, subject matter, or field of knowledge. In some cases, data domain 240 may include medical diagnostics, legal procedures, financial analysis, or engineering design. In some cases, data domain 240 may include both general vocabulary relevant to the topic and specialized terminology unique to that field.

In some embodiments, a data domain 240 may be created by grouping linguistic terms and their correlations according to metadata tags, keyword frequency patterns, semantic similarity measures, or predefined classification schemas. For the purposes of this disclosure, a “linguistic term” is a discrete unit of language that carries semantic meaning. In some cases, linguistic term may include a word, phrase, token, or symbol. In some cases, linguistic term may include natural language words from any human language, technical jargon specific to a subject matter domain, abbreviations, acronyms, alphanumeric identifiers, or multi-word expressions that function as a single semantic entity. As a non-limiting example, a linguistic term with a data domain 240 may include a term “antibiotic prophylaxis” associated with a medical data domain of “clinical pharmacology.” In some embodiments, a data domain 240 may be formed from curated datasets drawn from domain-specific sources, such as medical journals, legal codes, technical manuals, or structured databases.

In some cases, data domain 240 may be used in a training phase of a large language model 220 to focus learning on context-specific correlations, enabling the large language model 220 to generate outputs that are more accurate, relevant, and appropriate within the given domain. In some cases, data domains 240 may be selected, modified, or augmented as a function of feedback quality scores 248, allowing the system to improve domain-specific performance by emphasizing high-quality correlations and de-emphasizing low-quality or erroneous correlations within the same domain. In a non-limiting example, processor 104 may maintain an association between each general training set and one or more original data domains including domain-relevant linguistic terms. Upon receiving an advisory input and an associated feedback quality score 248 below a score threshold, processor 104 may determine that the original data domain 240 for general training set 236 is not optimally aligned with the advisory input. The processor 104 may then select an alternative data domain 240 from the plurality of data domains by analyzing semantic similarity between the advisory input and domain-specific linguistic terms, optionally weighted by historical model performance data. Once the alternative data domain 240 is selected, the processor 104 may modify the general training sets 236 by replacing, augmenting, or merging the original domain-specific linguistic terms with linguistic terms from the selected data domain 240. This modification may allow the updated general training sets to more accurately reflect the language, concepts, and correlations most relevant to the advisory input, thereby improving the LLM's ability to generate more accurate and context-appropriate therapeutic correctors in subsequent iterations. In some cases, user may manually determine data domain 240.

With continued reference to FIG. 2, in some cases, selecting data domain 240 may include extracting one or more linguistic terms associated with the selected data domain 240 from a plurality of data sources using a web crawling module 224e. For the purposes of this disclosure, a “data source” is any digital or physical repository, medium, or location from which textual content, linguistic terms, or other relevant information may be obtained. In some cases, data source may be structured, semi-structured, or unstructured. In some cases, data source may reside locally on a computing device, within a private network, or in a public or distributed environment such as the Internet. As a non-limiting example, a data source may include structured databases, spreadsheets, or knowledge graphs containing organized information about a specific data domain. In some embodiments, a data source may include unstructured or semi-structured text corpora, such as articles, research papers, technical manuals, legal documents, regulatory filings, medical guidelines, or online forum discussions. Data sources may also encompass dynamically generated or programmatically accessed content, such as web pages retrieved via a web crawler, APIs serving domain-specific content, or streaming data feeds. In some embodiments, data sources may include from web sources. For the purposes of this disclosure, a “web source” is any internet-based location or online resource that hosts or provides access to data. A web source may include, but is not limited to, websites, web pages, online databases, public or private APIs, social media platforms, forums, blogs, and news websites. For example, and without limitation, web source may include real estate platforms, local government property and building permit websites, and the like. In some embodiments, processor 104 may retrieve linguistic source from web sources using a web crawling module 224e.

With continued reference to FIG. 1, a “web crawling module,” as used herein, is a program that systematically browses the internet for the purpose of Web indexing. The web crawling module 224e may be seeded with platform URLs, wherein the web crawling module 224e may then visit the next related URL, retrieve the content, index the content, and/or measures the relevance of the content to the topic of interest. In some embodiments, processor 104 may generate web crawling module 224e to scrape linguistic terms associated with a particular data domain 240 from user's website. The web crawling module 224e may be seeded and/or trained with a reputable website to begin the search. Web crawling module 224e may be generated by processor 104. In some embodiments, web crawling module 224e may be trained with information received from user through a user interface. In some embodiments, web crawling module 224e may be configured to generate a web query. A web query may include search criteria received from user. For example, user may submit a plurality of websites for web crawling module 224e to search to linguistic terms associated with a particular data domain 240. Additionally, web crawling module 224e function may be configured to search for and/or detect one or more data patterns. A “data pattern” as used in this disclosure is any repeating forms of information. In some embodiments, web crawling module 224e may be configured to determine the relevancy of a data pattern. Relevancy may be determined by a relevancy score. A relevancy score may be automatically generated by processor 104, received from a machine learning model, and/or received from user. In some embodiments, a relevancy score may include a range of numerical values that may correspond to a relevancy strength of data received from a web crawling module function. As a non-limiting example, a web crawling module function may search the Internet for linguistic terms associated with a particular data domain 240.

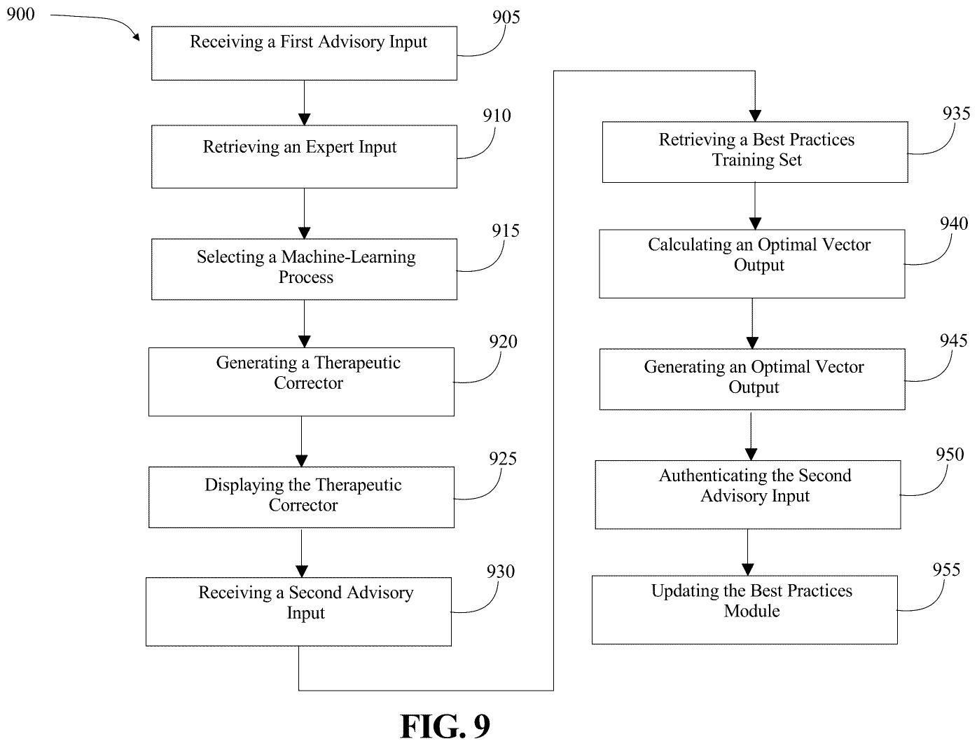

With continued reference to FIG. 1, in some cases, updating LLM 220 may include modifying specific training sets 244 by selecting one data cohort as a function of the advisory input and modifying the specific training sets 244 as a function of the selected data cohort. For the purposes of this disclosure, a “data cohort” is a subset of training data that is grouped together based on one or more shared attributes, characteristics, or contextual factors. As a non-limiting example, data cohort may be associated with a patient or advisor client. For example, and without limitation, data cohort may be associated with patient's or advisor client's medical condition, demographic characteristics, treatment history, laboratory results, or presenting symptoms. As a non-limiting example, data cohort may be associated with an expert. For example, and without limitation, data cohort may be associated with expert's professional specialty, years of experience, geographic region, or prior evaluation patterns when providing feedback on therapeutic correctors. In some embodiments, advisory input may be classified to a data cohort using a cohort classifier. Cohort classifier may be consistent with any classifier discussed in this disclosure. Cohort classifier may be trained on cohort training data, wherein the cohort training data may include advisory inquiries correlated to data cohorts. In some embodiments, advisory inquiries may be classified to data cohorts and processor 104 may determine data cohorts based on the user cohort using a machine-learning module as described in detail with respect to FIG. 9 and the resulting output may be used to update specific training sets 244.

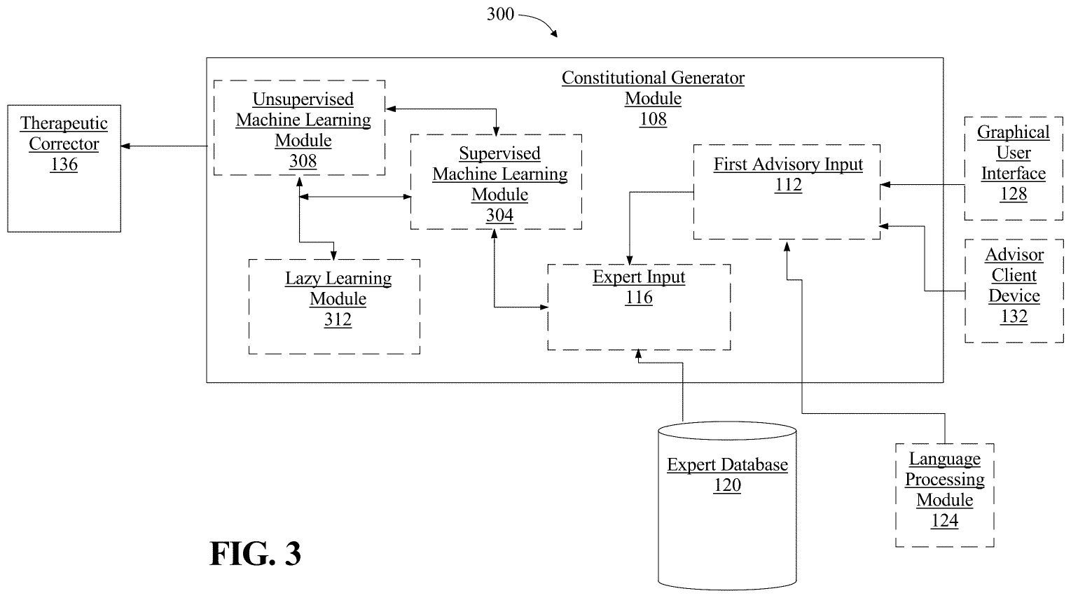

Referring now to FIG. 3, an exemplary embodiment 300 of constitutional generator module 108 is illustrated. Constitutional generator module 108 may be implemented as any hardware and/or software module. Constitutional generator module 108 receives a first advisory input 112 containing a constitutional inquiry and a user identifier. Constitutional generator module 108 may receive a first advisory input 112 from an input generated on graphical user interface 128. Constitutional generator module 108 may receive a first advisory input 112 from an input generated from an advisor client device. First advisory input 112 containing a constitutional inquiry may include any of the first advisory input 112 as described above in more detail in reference to FIG. 1. For example, first advisory input 112 may include a list of symptoms that a user may be experiencing and contain a request for a potential diagnosis. In yet another non-limiting example, first advisory input 112 may include a description of a series of symptoms that a user may be experiencing with a request to understand what tests an informed advisor should run to confirm or rule out potential diagnoses. In yet another non-limiting example, first advisory input 112 may include a question as to what tests an informed advisor should run to check progression of disease state such as small intestinal bacterial overgrowth (SIBO), which the informed advisor may be unfamiliar with treating.

With continued reference to FIG. 3, constitutional generator module 108 retrieves an expert input 116 from expert database 120 as a function of a first advisory input 112 and a user identifier. Expert input 116 may include any of the expert input 116 as described above in reference to FIG. 1. Expert input 116 may include an indication as to what machine-learning process constitutional generator module 108 may utilize to generate a therapeutic corrector 136 for a particular first advisory input 112. Constitutional generator module 108 may utilize language processing module 124 to extract one or more keywords contained within a first advisory input 112. For example, language processing module 124 may extract a string of words from a particular first advisory input 112 that contain a request for a potential diagnosis for a user who complains of symptoms that include fever, chills, and diarrhea. In such an instance, constitutional generator module 108 may utilize the string of words that contain user's symptoms to retrieve an expert input 116 that contains suggested training sets and/or machine-learning models that may be best to utilize with user's symptoms to generate a therapeutic corrector 136. In yet another non-limiting example, constitutional generator module 108 may utilize language processing module 124 to extract a particular diagnosis contained within a first advisory input 112 that may be utilized by constitutional generator module 108 to select an expert input 116 that contains suggested training sets that may be utilized to select a machine-learning process such as a supervised machine-learning model to output a therapeutic corrector 136 that contains suggested treatment options for the particular diagnosis contained within the first advisory input 112. Constitutional generator module 108 may utilize a user identifier to select an expert input 116 such as by utilizing a user identifier to review previous machine-learning models that were utilized to generate previous therapeutic corrector 136 for a user.

With continued reference to FIG. 3, constitutional generator module 108 may include supervised machine-learning module 304. Supervised machine-learning module 304 may be implemented as any hardware and/or software module. Supervised machine-learning module 304 may be configured to receive therapeutic training data from expert database 120. Therapeutic training data includes a plurality of data entries containing constitutional inquiries correlated to therapeutic corrector 136. For instance and without limitation, therapeutic training data may include constitutional inquiries that contain a list of symptoms correlated to therapeutic corrector 136 that contain potential diagnoses. One or more training sets may be stored in expert database 120. Therapeutic training data may be generated based on expert input 116, including any of the expert input 116 as described above. Supervised machine-learning module 304 may generate using a supervised machine-learning algorithm a therapeutic model that outputs a therapeutic corrector 136 utilizing the therapeutic training data and the advisory input containing the constitutional inquiry. Therapeutic model may include any machine learning process and may include linear or polynomial regression algorithms. Therapeutic model may include one or more equations. Therapeutic model may include a set of instructions utilized to generate outputs based on inputs derived using a machine-learning algorithm and the like. Therapeutic model may be utilized to generate a therapeutic corrector 136 that contains a response to a constitutional inquiry.

With continued reference to FIG. 3, constitutional generator module 108 may include unsupervised machine-learning module 308. Unsupervised machine-learning module may be implemented as any hardware and/or software module. Unsupervised machine-learning module may be configured to receive a plurality of unclassified data entries from expert database 120. “Unclassified data entries” as used in this disclosure, includes one or more data entries that have not been utilized in combination with one or more classification algorithms to generate one or more classification labels. Classification algorithms include any of the classification algorithms as described above including logistic regression, Naïve Bayes, decision trees, k-nearest neighbors, and the like. A “classification label” as used in this disclosure, includes any identification as to whether a particular data entries or series of data entries belong to a class or not. Classification may include the process of assigning a set of predefined categories or classes to one or more data entries utilizing classification algorithms. Predefined categories or classes may be generated and/or selected based on expert input 116, such as from expert database 120. Unsupervised machine-learning module may generate using an unsupervised machine-learning algorithm an unsupervised model that outputs a therapeutic correcting utilizing the plurality of unclassified data entries and the advisory input containing the constitutional inquiry. An unsupervised machine-learning process, as used herein, is a process that derives inferences in datasets without regard to labels; as a result, an unsupervised machine-learning process may be free to discover any structure, relationship, and/or correlation provided in the data. For instance, and without limitation, unsupervised machine-learning module 308 may perform an unsupervised machine learning process on plurality of unclassified data entries, which may cluster data contained within plurality of unclassified data entries according to detected relationships between elements of unclassified data entries, including without limitation correlations of elements of advisory inputs to each other and correlations of therapeutic corrector 136 to each other; such relations may then be combined with supervised machine learning results to add new criteria for supervised machine-learning module 304 to apply in relating advisory inputs to therapeutic corrector 136.

With continued reference to FIG. 3, constitutional generator module 108 may include lazy learning module 312. Lazy learning module may be implemented as any hardware and/or software module. A lazy-learning process and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover a “first guess” at a therapeutic corrector 136 associated with advisory inputs, using therapeutic training data. As a non-limiting example, an initial heuristic may include a ranking of therapeutic corrector 136 according to relation to a test type of an advisory input, one or more categories of therapeutic corrector 136 identified in test type of an advisory input, and/or one or more values detected in an advisory input; ranking may include, without limitation, ranking according to significance scores of associations between elements of advisory inputs and therapeutic corrector 136, for instance as calculated as described above. Heuristic may include selecting some number of highest-ranking associations and/or therapeutic corrector 136. Lazy learning module may alternatively or additionally implement any suitable “lazy learning” algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate therapeutic corrector 136 as described in this disclosure, including without limitation lazy learning applications of machine-learning algorithms as described in further detail below.

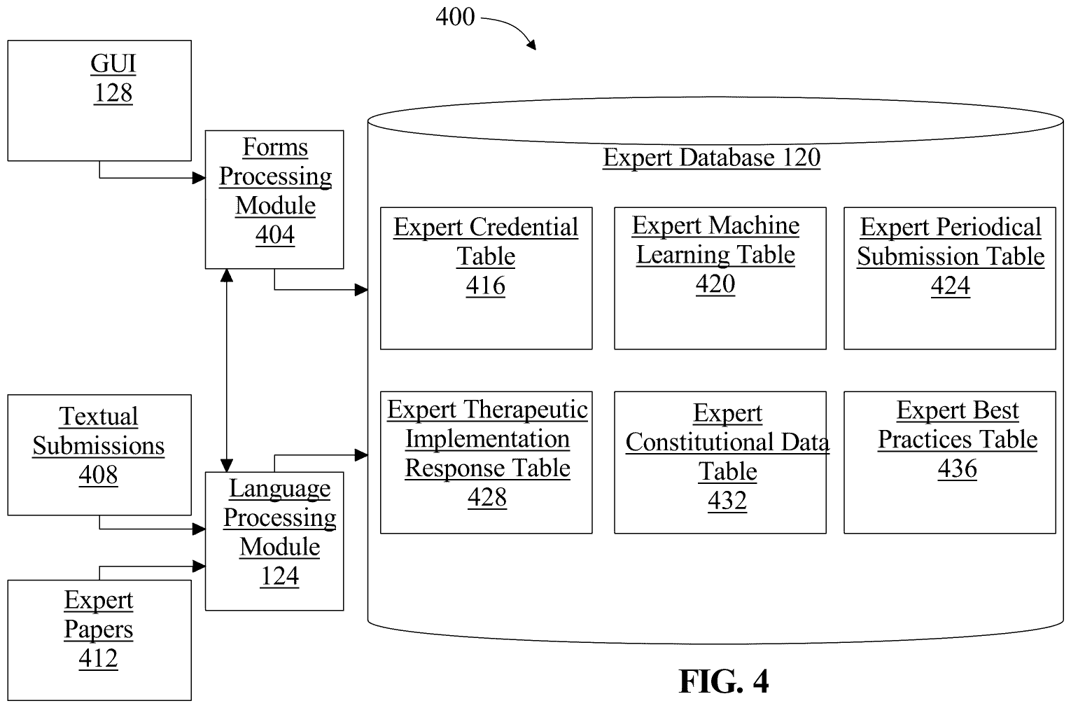

Referring now to FIG. 4, an exemplary embodiment 400 of expert database 120 is illustrated. Expert database 120 may be implemented as any data structure as described above in reference to FIG. 1. One or more database tables may be linked to one another by, for instance, common column values. For instance, a common column between two tables of expert database 120 may include an identifier of an expert submission, such as a form entry, textual submission, expert paper, or the like, for instance as defined below; as a result, a query may be able to retrieve all rows from any table pertaining to a given submission or set thereof. Other columns may include any other category usable for organization or subdivision of expert data, including types of expert data, names and/or identifiers of experts submitting the data, times of submission, or the like; persons skilled in the art, upon reviewing the entirety of this disclosure, will be aware of various ways in which expert data may be included in one or more tables.

With continued reference to FIG. 4, expert database 120 includes a forms processing module 404 that may sort data entered in a submission via graphical user interface 128 by, for instance, sorting data from entries in the graphical user interface 128 to related categories of data; for instance, data entered in an entry relating in the graphical user interface 128 to a clustering algorithm may be sorted into variables and/or data structures for storage of clustering algorithms, while data entered in an entry relating to a category of training data and/or an element thereof may be sorted into variables and/or data structures for the storage of, respectively, categories of training data. Where data is chosen by an expert from pre-selected entries such as drop-down lists, data may be stored directly; where data is entered in textual form, language processing module 124 may be used to map data to an appropriate existing label, for instance using a vector similarity test or other synonym-sensitive language processing test to map physiological data to an existing label. Alternatively or additionally, when a language processing algorithm, such as vector similarity comparison, indicates that an entry is not a synonym of an existing label, language processing module 124 may indicate that entry should be treated as relating to a new label; this may be determined by, e.g., comparison to a threshold number of cosine similarity and/or other geometric measures of vector similarity of the entered text to a nearest existent label, and determination that a degree of similarity falls below the threshold number and/or a degree of dissimilarity falls above the threshold number. Data from expert textual submissions 408, such as accomplished by filling out a paper or PDF form and/or submitting narrative information, may likewise be processed using language processing module 124. Data may be extracted from expert papers 412, which may include without limitation publications in medical and/or scientific journals, by language processing module 124 via any suitable process as described herein. Persons skilled in the art, upon reviewing the entirety of this disclosure, will be aware of various additional methods whereby novel terms may be separated from already-classified terms and/or synonyms therefore, as consistent with this disclosure.

With continued reference to FIG. 4, one or more tables contained within expert database 120 may include expert credential table 416; expert credential table 416 may include one or more data entries relating to expert credentials. One or more tables contained within expert database 120 may include expert machine learning table 420; expert machine learning table 420 may include one or more data entries relating to machine learning include training sets, machine-learning algorithms, supervised machine-learning processes, unsupervised machine-learning processes and the like. One or more tables contained within expert database 120 may include expert periodical submission table 424; expert periodical submission table 424 may include one or more data entries relating to expert periodical submissions. One or more tables contained within expert database 120 may include expert therapeutic implementation response 428; expert therapeutic implementation response 428 may include one or more data entries relating to expert therapeutic implementation responses. One or more tables contained within expert database 120 may include expert constitutional data table 432; expert constitutional data table 432 may include one or more data entries relating to constitutional data. One or more tables contained within expert database 120 may include expert best practices table 436; expert best practices table 436 may include one or more data entries relating to best practices.

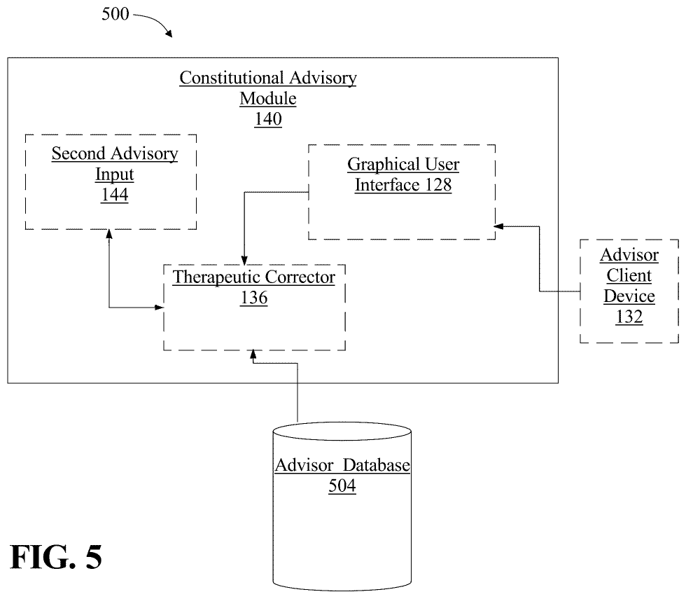

Referring now to FIG. 5, an exemplary embodiment 500 of constitutional advisory module 140 is illustrated. Constitutional advisory module 140 may be implemented as any hardware and/or software module. Constitutional advisory module 140 is configured to receive a therapeutic corrector 136 from constitutional generator module 108. Constitutional advisory module 140 may receive a therapeutic corrector 136 from constitutional generator module 108 utilizing any network methodology as described herein. Constitutional advisory module 140 displays the therapeutic corrector 136 on graphical user interface 128 located on processor 104. In an embodiment, constitutional advisory module 140 may display the therapeutic corrector 136 to a particular informed advisor who generated a first advisory input 112. Constitutional advisory module 140 receives a second advisory input 144 from an advisor client device operated by an informed advisor wherein the second advisory input contains a therapeutic corrector implementation response. Second advisory input 144 contains a therapeutic corrector implementation response. Therapeutic corrector implementation response may include any of the therapeutic corrector implementation responses as described above in reference to FIG. 1. For example, second advisory input 144 may include an informed advisor's description regarding implementing a particular treatment regimen for a user suggested within a therapeutic corrector 136. One or more advisory inputs may be stored in advisor database 504. Advisory database may include any data structure suitable for use as expert database 120 as described above.

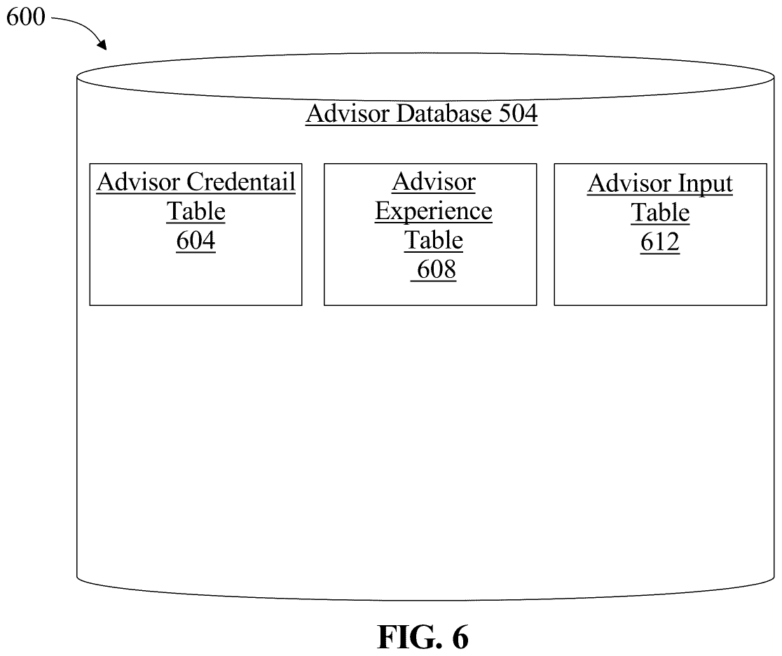

Referring now to FIG. 6, an exemplary embodiment 600 of advisor database 504 is illustrated. One or more tables contained within advisor database 504 may include advisor credential table 604; advisor credential table 604 may include one or more data entries describing an informed advisor's credentials. For instance and without limitation, credential table 604 may include information pertaining to an informed advisor's board certifications, education, clinical training, clinical experience, publications, licenses, and the like. One or more tables contained within advisor database 504 may include advisor experience table 608; advisor experience table 608 may include one or more data entries describing an informed advisor's clinical experience. For example, advisor experience table 608 may include information describing an informed advisor's clinical practice including number of patients treated each year, clinical success, medical conditions treated and the like. One or more tables contained within advisor database 504 may include advisor input table 612; advisor input table 612 may include one or more data entries describing inputs generated by an informed advisor. For example, advisor input table 612 may include stored data entries containing a first advisor input and a second advisor input.

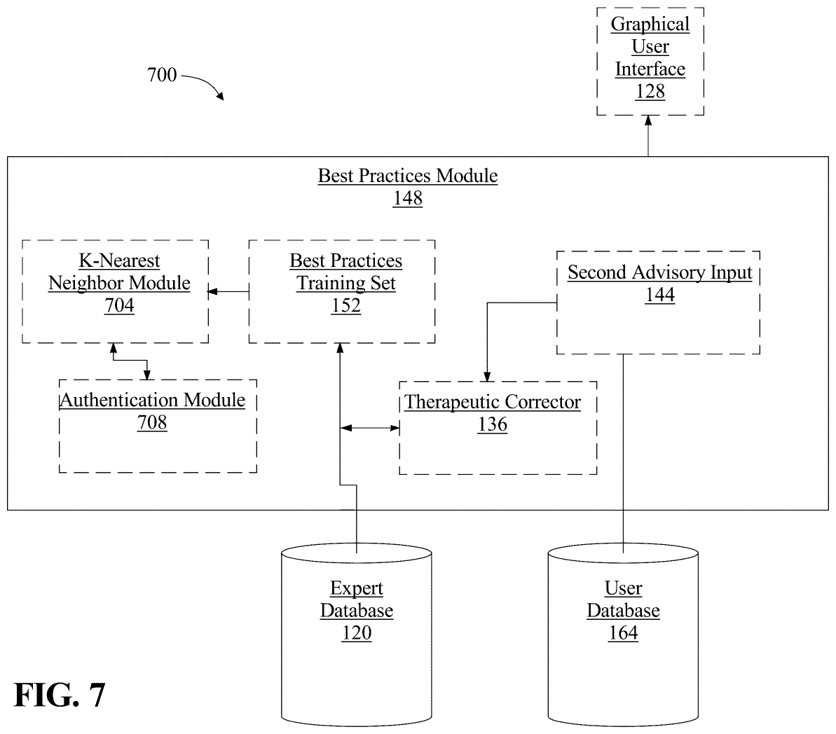

Referring now to FIG. 7, an exemplary embodiment 700 of best practices module 148 is illustrated. Best practices module 148 may be implemented as any hardware and/or software module. Best practices module 148 receives the second advisory input 144 containing the therapeutic corrector implementation response from the constitutional advisory module 140. This may be performed utilizing any network methodology as described herein. Best practices module 148 receives the therapeutic corrector 136 from the constitutional generator module 108. This may be performed utilizing any network methodology as described herein.

With continued reference to FIG. 7, best practices module 148 retrieves from expert database 120 a best practices training set 152. Best practices training set 152 correlates a therapeutic corrector 136 to therapeutic corrector implementation responses. Best practice training set may be generated based on input from one or more experts as described above. For example, best practices training set 152 may correlate a therapeutic corrector 136 such as initiating a fish oil supplement to one or more therapeutic corrector implementation responses that include decreased triglyceride levels, decreased total cholesterol levels, and increased high density lipoprotein (HDL) levels. In yet another non-limiting example, best practice training set may correlate a therapeutic corrector 136 such as initiation of a meditation sequence for a user with major depressive disorder with one or more therapeutic corrector implementation responses that include reduced number of depressive episodes, decreased nights experiencing insomnia, increased energy, and increased attendance at social gatherings.

With continued reference to FIG. 7, best practices module 148 may include k-nearest neighbor module 704. K-nearest neighbor module 704 may be implemented as any hardware and/or software module. K-nearest neighbor module may calculate an optimal vector output for the therapeutic corrector 136 received from the constitutional generator module 108 utilizing a k-nearest neighbor algorithm 156 and the best practices training set 152. K-nearest neighbor module may modify best practices training set 152 by representing best practices training set 152 as vectors. Vectors may include mathematical representations of best practices training set 152. Vectors may include n-tuple of values which may represent a measurement or other quantitative value associated with a given category of data, or attribute. Vectors may be represented in n-dimensional space using an axis per category of value represented in n-tuple of values, such that a vector has a geometric direction characterizing the relative quantities of attributes in the n-tuple as compared to each other. In an embodiment, K-nearest neighbor module may calculate an initial heuristic ranking association between therapeutic corrector 136 and elements of best practices training set 152. Initial heuristic may include selecting some number of highest-ranking associations and/or training set elements. K-nearest neighbor module may perform one or more processes to modify and/or format classified best practices training set 152. Classified best practices training set 152 may contain “N” unique features, whereby a dataset contained within classified best practices training set 152 and represented as a vector may contain a vector of length “N” whereby entry “I” of the vector represents that data point's value for feature “I.” Each vector may be mathematically represented as a point in “R N.” For instance and without limitation, K-nearest neighbor module may modify entries contained within classified best practices training set 152 training data to contain consistent forms of a variance. After appropriate selection of best practices training set 152, K-nearest neighbor module performs K-nearest neighbors algorithm by classifying therapeutic datasets contained within the selected classified best practices training set 152. Selected classified best practices training set 152 training data may be represented as an “M×N” matrix where “M” is the number of data points contained within the classified best practices training set 152 training data and “N” is the number of features contained within the selected classified best practices training set 152 training data. Classifying datasets contained within selected classified best practices training set 152 training data set may include computing a distance value between an item to be classified such as a therapeutic dataset and each dataset contained within selected classified best practices training set 152 training set which may be represented as a vector. A value of “k” may be pre-determined or selected that will be used for classifications. In an embodiment, value of “k” may be selected as an odd number to avoid a tied outcome. In an embodiment, value of “k” may be decided by K-nearest neighbor module arbitrarily or value may be cross validated to find an optimal value of “k.”. K-nearest neighbor module may then select a distance metric that will be used in K-nearest neighbors algorithm. In an embodiment, K-nearest neighbor module may utilize Euclidean distance which may be measure distance by subtracting the distance between a training data point and the datapoint to be classified such as a therapeutic corrector 136. In an embodiment, Euclidean distance may be calculated by a formula represented as: E(x,y)=\sqrt{Σᵢ₌₀ⁿ(xi−yi)²}. In an embodiment, K-nearest neighbor module may utilize metric distance of cosine similarity which may calculate distance as the difference in direction between two vectors which may be represented as: similarity=cos 0=A×B÷∥A∥∥B∥. After selection of “k” value, and selection of distance measurement by K-nearest neighbor module, K-nearest neighbor module may partition in “R\hat{ }N” into sections. Sections may be calculated using the distance metric and the available data points contained within selected classified best practices training set 152. K-nearest neighbor module may calculate a plurality of optimal vector outputs; in such an instance, where a plurality of matching entries is returned, optimal vector output may be obtained by aggregating matching entries including any suitable method for aggregation, including component-wise addition followed by normalization component-wise calculation of arithmetic means, or the like. K-nearest neighbor module generates an optimal vector output containing an expected therapeutic corrector implementation response 160.

With continued reference to FIG. 7, best practices module 148 may include authentication module 708. Authentication module 708 may be implemented as any hardware and/or software module. Authentication module 708 authenticates a second advisory input 144 containing a therapeutic corrector implementation response based on an expected therapeutic corrector implementation response 160 generated by k-nearest neighbor module 704. Authentication module 708 may authenticate a second advisory input 144 containing a therapeutic corrector implementation response by determining if a therapeutic corrector implementation response matches one or more expected therapeutic corrector implementation response 160 generated by k-nearest neighbor module 704. For example, authentication module 708 may receive second advisory input 144 from constitutional advisory module 140 that contains a therapeutic corrector implementation response that contains a description of a side effect an informed advisor noticed when a user started on a nutritional supplement that included the development of a dry cough. In such an instance, authentication module 708 may compare the side effect of a dry cough to one or more expected therapeutic corrector implementation response 160 generated by k-nearest neighbor module 704 includes a dry cough. If for example, dry cough was listed as one or more expected therapeutic corrector implementation response 160, then authentication module 708 may authenticate the second advisory input 144 containing the therapeutic corrector implementation response. In such an instance, authentication module 708 may then update the best practices module 148 to incorporate the therapeutic corrector implementation response as a data entry in expert database 120, as a training set entry in best practices training set 152, and/or incorporating the therapeutic corrector implementation response into a particular machine-learning model.

With continued reference to FIG. 7, authentication module 708 may authenticate a therapeutic corrector implementation response that does not match an expected therapeutic implementation response generated by k-nearest neighbor module 704 by obtaining a second authenticator, to determine if a particular therapeutic corrector implementation response may include an observation or response that may not be widely known or accepted by the medical community yet. For example, an informed advisor may have success utilizing a known medication for a new use in a user with a rare disease that may have never been observed by any other experts and may not be generated as an expected therapeutic implementation response. In such an instance, authentication module 708 may be unable to authenticate the therapeutic corrector implementation response and may have to seek to authenticate the therapeutic corrector implementation response by other measures including verifying expert periodicals to determine if there has been any other literature available that describes the new use for the known medication in a rare genetic disease, or obtaining a second opinion from a second informed advisor who may also be a known expect in a field relating to a therapeutic corrector implementation response, or examining user constitutional data to determine if a user reports feeling better since starting the new medication or if any health measurements of a user have improved.

With continued reference to FIG. 7, authentication module 708 may authenticate a therapeutic corrector implementation response by receiving a second expected therapeutic corrector implementation response 160 from a second informed advisor. Authentication module 708 may display the second advisory input 144 containing a therapeutic corrector implementation response and an expected therapeutic corrector implementation response 160 on a graphical user interface 128 located on processor 104 to a second informed advisor. Second informed advisor may include any informed advisor other than first informed advisor. Second informed advisor may be of a particular specialty and may practice in an area of medicine or healthcare similar to first informed advisor. Second informed advisor may also be considered a specialist in regard to the topic of a particular therapeutic corrector implementation response. Authentication module 708 may select a second informed advisor to receive a second expected therapeutic corrector implementation response 160 by locating a potential second informed advisor's credentials from advisor database 504 or looking for a potential second informed advisor who has a certain level of experience or knowledge which may be stored within advisor database. Second informed advisor may generate a second expected therapeutic corrector implementation response 160 which authentication module 708 may receive and utilize to authenticate the second advisory input 144. Authentication module 708 may authenticate a second informed advisor's credentials by receiving a second expert credential validator from second informed advisor. Second expert credential validator may include any expert credential validator as described above in reference to FIG. 1. Authentication module 708 may compare a second expert credential validator to a list of known expert credentials stored in expert database 120 and determine that the second expert credential validator is authentic.

With continued reference to FIG. 7, authentication module 708 may authenticate a therapeutic corrector implementation response by determining if a potential therapeutic corrector implementation response is contained within a particular periodical submission. Authentication module 708 may retrieve an expert periodical submission contained within expert database 120 and locate an expected therapeutic corrector implementation response 160 contained within the expert periodical submission. In an embodiment, expert periodical submissions contained within expert database 120 may be organized according to particular categories and/or topics so that locating particular entries relating to particular topics contained within therapeutic corrector implementation responses may be performed rapidly and with ease. In an embodiment, language processing module 124 may be utilized to locate one or more words or strings of words that may be extracted from a particular therapeutic corrector implementation response that may summarize the topic or field of medicine that the therapeutic corrector implementation response relates to. Such words and/or strings of words may be utilized to located expert periodical submission that are related to the topic or field of medicine contained within the words or strings of words. Authentication module 708 may compare a therapeutic corrector implementation response to a second expected therapeutic corrector implementation response 160 contained within an expert periodical submission. Authentication module 708 may then utilize a second expected therapeutic corrector implementation response 160 to authenticate a first therapeutic implementation response if a second expected therapeutic corrector implementation response 160 matches an expected therapeutic implementation response. A therapeutic corrector implementation response that is authenticated such as by matching to a second expected therapeutic corrector implementation response 160 may be utilized to update best practices module 148 such as by incorporating a therapeutic corrector implementation response into expert database 120 and/or incorporating the second expected therapeutic implementation response into a training set.

With continued reference to FIG. 7, authentication module 708 may authenticate a second advisory input 144 containing a therapeutic corrector implementation response by comparing the therapeutic corrector implementation response to user constitutional data. Authentication module 708 may retrieve an element of user constitutional data from user database 164. User constitutional data may include any of the user constitutional data as described above. For example, user constitutional data may include one or more physically extracted samples such as a hair sample analyzed for heavy metals or a urine sample analyzed for the presence or absence of ketones. User constitutional data may also include one or more descriptions of how a user may be feeling or responding to particular treatment. For example, user constitutional data may include medical records containing subjective and objective assessments of a user, such as from consultations and appointments with functional medicine doctors who may report how a user is feeling based on in-person or phone calls discussing a user's progress with a particular diagnosis or treatment. User constitutional data may include user reports such as self-assessments or journal entries describing how a user feels or is feeling in regard to particular medical intervention, diagnosis, treatment, and the like. Authentication module 708 may retrieve an element of user constitutional data from a user database 164 and compare an element of user constitutional data to a therapeutic corrector implementation response. For example, a therapeutic corrector implementation response that contains a description of a user who experienced unknown side effects from an acupuncture treatment such as ringing in the ears may be utilized to locate an element of user constitutional data contained within user database 164 to determine if user ever complained of ringing in the ears during an acupuncture treatment. In yet another non-limiting example, a therapeutic corrector implementation response that contains a description of a user who experienced remission of user's diabetes after being treatment for a medication intended to treat user's hepatitis, may be utilized to extract an element of user constitutional data that includes user's most recent fasting blood glucose and hemoglobin A1C results to determine if user's blood sugars accurately show that user's diabetes is in fact in remission. A therapeutic corrector implementation response that is authenticated by an element of user constitutional data may be utilized to update expert knowledge module utilizing any of the methods as described above.

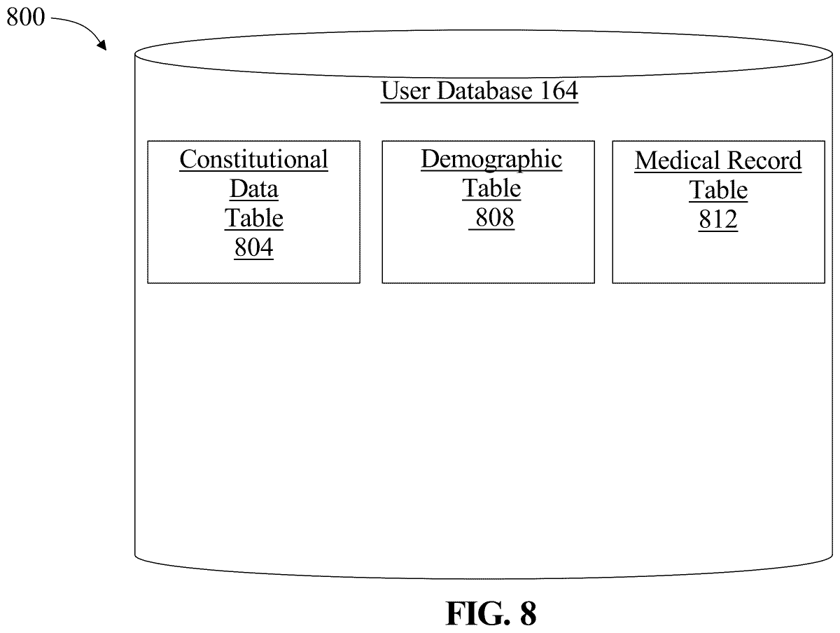

Referring now to FIG. 8, an exemplary embodiment 800 of user database 164 is illustrated. User database 164 may be implemented as any data structure suitable for use as expert database 120 as described above in reference to FIG. 1. One or more tables contained within user database 164 may include constitutional data table 804; constitutional data table 804 may include one or more data entries containing an element of user constitutional data. For example, constitutional data table 804 may include one or more results from a medical imaging test, one or more analyzed blood samples analyzed for intracellular and extracellular nutrient levels, one or more saliva samples analyzed for hormone levels and the like. One or more tables contained within user database 164 may include demographic table 808; demographic table 808 may include one or more data entries containing user demographic information. For example, demographic table 808 may include information describing user's full legal name, address, occupation, education level, marital status, and the like. One or more tables contained within user database 164 may include medical record table 812; medical record table 812 may include one or more data entries describing a user's medical records. For example, medical record table 812 may include one or more clinical notes from an appointment with an informed advisor, one or more subjective findings from a physical exam, one or more notes summarizing how a user feels and the like.

Referring now to FIG. 9, an exemplary embodiment of a machine-learning module 900 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 904 to generate an algorithm instantiated in hardware or software logic, data structures, and/or functions that will be performed by a computing device/module to produce outputs 908 given data provided as inputs 912; this is in contrast to a non-machine learning software program where the commands to be executed are determined in advance by a user and written in a programming language.

Still referring to FIG. 9, “training data,” as used herein, is data containing correlations that a machine-learning process may use to model relationships between two or more categories of data elements. For instance, and without limitation, training data 904 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 904 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 904 according to various correlations; correlations may indicate causative and/or predictive links between categories of data elements, which may be modeled as relationships such as mathematical relationships by machine-learning processes as described in further detail below. Training data 904 may be formatted and/or organized by categories of data elements, for instance by associating data elements with one or more descriptors corresponding to categories of data elements. As a non-limiting example, training data 904 may include data entered in standardized forms by persons or processes, such that entry of a given data element in a given field in a form may be mapped to one or more descriptors of categories. Elements in training data 904 may be linked to descriptors of categories by tags, tokens, or other data elements; for instance, and without limitation, training data 904 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 9, training data 904 may include one or more elements that are not categorized; that is, training data 904 may not be formatted or contain descriptors for some elements of data. Machine-learning algorithms and/or other processes may sort training data 904 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 904 to be made applicable for two or more distinct machine-learning algorithms as described in further detail below. Training data 904 used by machine-learning module 900 may correlate any input data as described in this disclosure to any output data as described in this disclosure. As a non-limiting illustrative example, input data may include advisory input, constitutional inquiry, feedback input, adherence input, and the like. As a non-limiting illustrative example, output data may include first therapeutic corrector, second therapeutic corrector, therapeutic corrector, feedback quality score, and the like.

Further referring to FIG. 9, training data may be filtered, sorted, and/or selected using one or more supervised and/or unsupervised machine-learning processes and/or models as described in further detail below; such models may include without limitation a training data classifier 916. Training data classifier 916 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 900 may generate a classifier using a classification algorithm, defined as a processes whereby a computing device and/or any module and/or component operating thereon derives a classifier from training data 904. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 916 may classify elements of training data to data cohorts related to a patient, advisor client, expert, and the like.

Still referring to FIG. 9, computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)−P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 9, computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 9, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 9, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 9, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 9, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 9, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 9, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 9, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 9, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 9, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mea}}{σ} .$$

Scaling may be performed using a median value of a set or subset X_{median} and/or interquartile range (IQR), which represents the difference between the 25th percentile value and the 50^{th} percentile value (or closest values thereto by a rounding protocol), such as:

$${X}_{new} = \frac{X - {X}_{median}}{IQR} .$$

Persons skilled in the art, upon reviewing the entirety of this disclosure, will be aware of various alternative or additional approaches that may be used for feature scaling.

Further referring to FIG. 9, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 9, machine-learning module 900 may be configured to perform a lazy-learning process 920 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 904. Heuristic may include selecting some number of highest-ranking associations and/or training data 904 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 9, machine-learning processes as described in this disclosure may be used to generate machine-learning models 924. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 924 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 924 may be generated by creating an artificial neural network, such as a convolutional neural network comprising an input layer of nodes, one or more intermediate layers, and an output layer of nodes. Connections between nodes may be created via the process of “training” the network, in which elements from a training data 904 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 9, machine-learning algorithms may include at least a supervised machine-learning process 928. At least a supervised machine-learning process 928, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include advisory input, constitutional inquiry, feedback input, adherence input, and the like as described above as inputs, first therapeutic corrector, second therapeutic corrector, therapeutic corrector, feedback quality score, and the like as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 904. Persons skilled in the art, upon reviewing the entirety of this disclosure, will be aware of various possible variations of at least a supervised machine-learning process 928 that may be used to determine relation between inputs and outputs. Supervised machine-learning processes may include classification algorithms as defined above.

With further reference to FIG. 9, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 9, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 9, machine learning processes may include at least an unsupervised machine-learning processes 932. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 932 may not require a response variable; unsupervised processes 932 may be used to find interesting patterns and/or inferences between variables, to determine a degree of correlation between two or more variables, or the like.

Still referring to FIG. 9, machine-learning module 900 may be designed and configured to create a machine-learning model 924 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 9, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 9, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 9, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 9, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 9, one or more processes or algorithms described above may be performed by at least a dedicated hardware unit 936. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 936 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 936 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 936 to perform one or more operations described herein, such as evaluation of model and/or algorithm outputs, one-time or iterative updates to parameters, coefficients, weights, and/or biases, and/or any other operations such as vector and/or matrix operations as described in this disclosure.

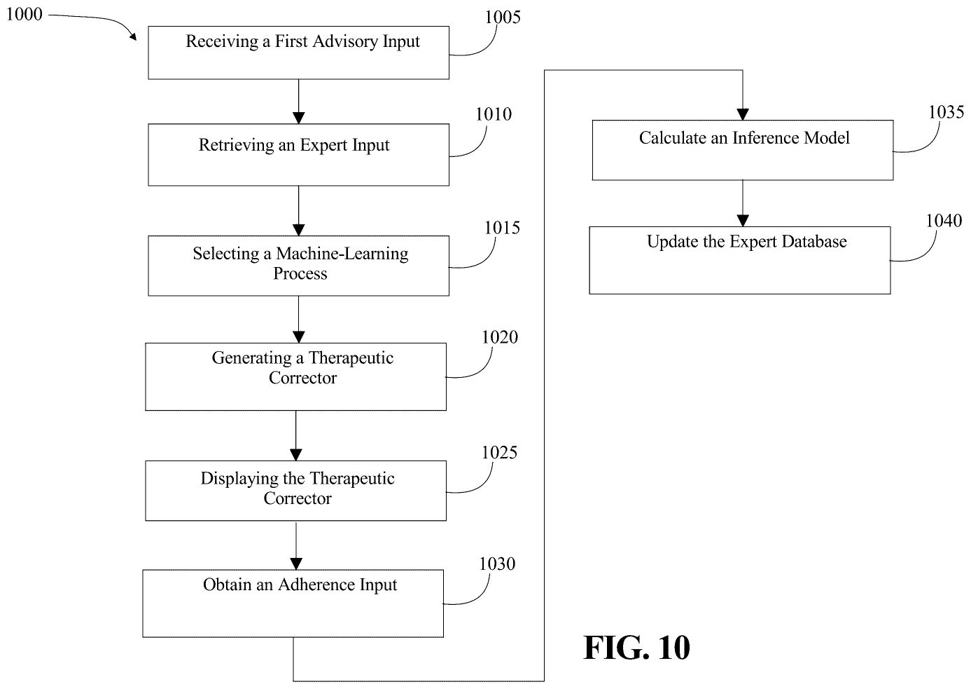

Referring now to FIG. 10, an exemplary embodiment of neural network 1000 is illustrated. A neural network 1000 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 1004, one or more intermediate layers 1008, and an output layer of nodes 1012. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

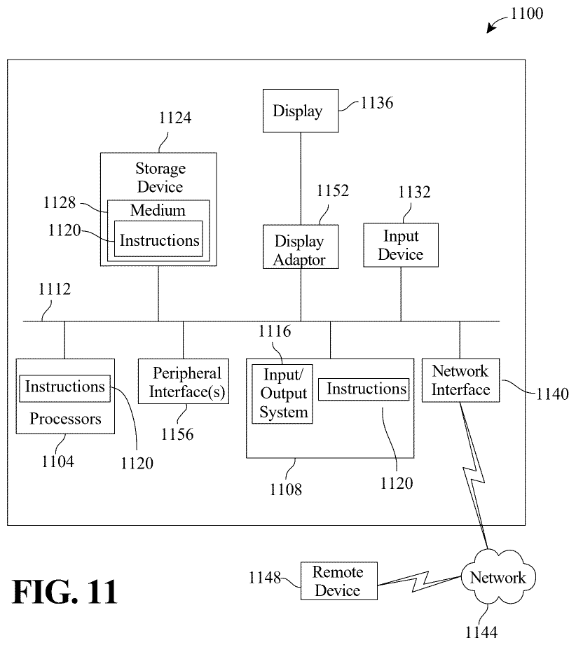

Referring now to FIG. 11, an exemplary embodiment of a node 1100 of a neural network is illustrated. A node may include, without limitation, a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0,x), a “leaky” and/or “parametric” rectified linear unit function such as f(x)=max(ax,x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ, or of other coefficients and/or parameters of an activation function, may be determined by training a neural network using training data, which may be performed using any suitable process as described above. Each weight in a neural network may, without limitation, be updated and/or tuned, based on an error function J, using a backpropagation updating method, such as:

$${w}_{new} = {w}_{old} - α \frac{dJ}{dw}$$

where w_{new} is the updated weight value, w_{old} is the previous weight value, α is a parameter to set the learning rate, and

$$\frac{dJ}{dw}$$

is the partial derivative of with respect to weight w

Referring now to FIG. 12, an exemplary embodiment 1200 of a method for confirming an advisory interaction with an artificial intelligence platform is illustrated. Method contains a step 1205 of receiving, using at least a processor, an advisory input including a constitutional inquiry. These may be implemented as reference to FIGS. 1-11.

With continued reference to FIG. 12, method contains a step 1210 of generating, using at least a processor, a first therapeutic corrector as a function of an advisory input using a large language model (LLM) and one or more machine-learning modules, wherein the LLM has been trained with first LLM training data including correlations between keywords. In some embodiments, the LLM has been generally trained with general training sets of the first LLM training data, wherein the general training sets may include correlations between one or more linguistic terms associated with a particular data domain and specifically trained with specific training sets of the first LLM training data, wherein the specific training sets may include exemplary advisory inputs correlated to exemplary therapeutic correctors. In some embodiments, generating the first therapeutic corrector may include determining at least a treatment using a first machine-learning module of the one or more machine-learning modules that has been trained with first training data including exemplary advisory inputs correlated to exemplary treatments. In some embodiments, generating the first therapeutic corrector may include determining at least a diagnosis using a second machine-learning module of the one or more machine-learning modules that has been trained with second training data including exemplary advisory inputs correlated to exemplary diagnoses. In some embodiments, generating the first therapeutic corrector may include determining at least a lab work using a third machine-learning module of the one or more machine-learning modules that has been trained with third training data including exemplary advisory inputs correlated to exemplary lab works. These may be implemented as reference to FIGS. 1-11.

With continued reference to FIG. 12, method contains a step 1215 of receiving, using at least a processor, a feedback input in response to an implementation of a first therapeutic corrector, wherein at least a part of the feedback input indicates that the first therapeutic corrector is incorrect. These may be implemented as reference to FIGS. 1-11.

With continued reference to FIG. 12, method contains a step 1220 of generating, using at least a processor, a feedback quality score as a function of a feedback input using a scoring machine-learning model of one or more machine-learning modules that has been trained with scoring training data including exemplary feedback inputs correlated to exemplary feedback quality scores. In some embodiments, generating the feedback quality score may include receiving an adherence input from a monitoring device, and generating the feedback quality score as a function of the feedback input and the adherence input. These may be implemented as reference to FIGS. 1-11.

With continued reference to FIG. 12, method contains a step 1225 of updating, using at least a processor, a LLM and one or more machine-learning modules as a function of at least in part on a feedback quality score, wherein updating includes generating second LLM training data including first LLM training data and therapeutic correctors that are incorrectly generated using the first LLM training data and generating a second therapeutic corrector using the updated LLM that is retrained with the second LLM training data. In some embodiments, updating the LLM may include modifying the specific training sets by generating a synthetic data pair incorporating the advisory input and the first therapeutic corrector with the feedback quality score lower than a score threshold, and augmenting the specific training sets with the synthetic data pair, wherein augmenting the specific training sets may include re-weighting one or more existing data pairs in the specific training sets that include correlations related to correlations in the synthetic data pair based on the feedback quality score. In some embodiments, updating the LLM may include modifying the general training sets by selecting one data domain from a plurality of data domains as a function of the advisory input and the feedback quality score, and modifying the general training sets to include one or more linguistic terms including the selected data domain. In some embodiments, selecting the one data domain may include extracting the one or more linguistic terms associated with the selected data domain from a plurality of data sources using a web crawling module. In some embodiments, updating the LLM may include modifying the specific training sets by selecting one data cohort as a function of the advisory input, and modifying the specific training sets as a function of the selected data cohort. These may be implemented as reference to FIGS. 1-11.

With continued reference to FIG. 12, method contains a step 1230 of generating, using at least a processor, a graphical user interface displaying a second therapeutic corrector. These may be implemented as reference to FIGS. 1-11.

Referring now to FIG. 13, an exemplary embodiment 1300 of another method of confirming an advisory interaction with an artificial intelligence platform is illustrated. At step 1305 a processor 104 receives a first advisory input 112 containing a constitutional inquiry and a user identifier. Processor 104 may receive a first advisory input 112 utilizing any network methodology as described herein. Processor 104 may receive a first advisory input containing a constitutional inquiry and a user identifier from an advisor client device operated by an informed advisor. A first advisory input 112 contains a constitutional inquiry. Constitutional inquiry may include any of the constitutional inquires as described above in reference to FIGS. 1-12. A constitutional inquiry may include any inquiry pertaining to the human body generated by an informed advisor. An informed advisor may include any of the informed advisors as described above in reference to FIGS. 1-12. For example, a constitutional inquiry may include an inquiry as to possible diagnoses for a user who may be suffering from symptoms that include back ache, fatigue, and muscle spasms. In yet another non-limiting example, a constitutional inquiry may include an inquiry as to possible treatments an informed advisor should consider when treating a particular illness the informed advisor may be unfamiliar with treating or may have not treated in a long time and may be somewhat perplexed as to where the informed advisor should initiate treatment. First advisory input 112 contains a user identifier. User identifier may include any of the user identifiers as described above in reference to FIGS. 1-12.

With continued reference to FIG. 13, a processor 104 may validate an informed advisor's credentials upon receiving a first advisory input 112. In conjunction with receiving a first advisory input 112, a processor 104 may receive a first expert credential validator. First expert credential validator may include any of the first expert credential validators as described above in reference to FIGS. 1-12. Processor 104 may compare a first expert credential validator to a list of known expert credentials stored in an expert database 120. Processor 104 may determine that the first expert credential validator is authentic upon locating the first expert credential validator on the expert list.

With continued reference to FIG. 13, at step 1310 a processor 104 retrieves an expert input 116 from best practices module 148 operating on the processor 104 as a function of a first advisory input 112 and a user identifier. Expert input 116, includes any expert submission as described above in more detail in reference to FIG. 1. Expert input 116 may be generated by one or more informed advisors who may be considered experts in a particular field of medicine and/health care such as by demonstrating certain professional milestones, having particular credentials and/or licenses, passing specific certifying exams, publishing articles, papers, and/or journal submissions on particular topics and the like. Expert input 116 may include expert advice as to particular training sets that can be selected based on particular first advisory input 112 or particular machine-learning models that may be best suited to be calculated for particular first advisory input 112. For instance and without limitation, expert input 116 may dictate that a first advisory input 112 containing a request for a list of particular diagnoses based on specific symptoms may be best suited to a supervised machine-learning algorithm while a first advisory input 112 containing a request for a list of possible treatments for a specific medical condition may be best suited for a lazy-learning algorithm such as k-nearest neighbor. Expert input 116 may be stored within expert database 120, which may include any data structure as described in more detail above. Expert input 116 may be constantly updated in real time to account for new discoveries and new research that may be published. Further, expert input 116 may be revoked such as when an expert's credentials may lapse, or an expert may suddenly die, or subsequent research comes out that invalidates previously demonstrate research. In an embodiment, expert input 116 may be retrieved based on previous interactions with a user and system 100. Processor 104 may utilize a user identifier to retrieve information from user database 164 that may contain information about previous expert input 116 utilized in reference to a particular user.

With continued reference to FIG. 13, at step 1315 a processor 104 selects a machine-learning process as a function of an expert input 116. A machine-learning process may include any of the machine-learning processes as described above in reference to FIGS. 1-12. Machine-learning processes may include supervised machine-learning processes, unsupervised machine-learning processes, lazy-learning processes and the like. One or more machine-learning processes, machine-learning models, and/or training sets may be stored within expert database 120. Processor 104 may select a particular machine-learning process based on expert input 116. For example, expert input 116 may describe a particular machine-learning model that may be best suited to generate specific therapeutic corrector 136 identified within a first advisory input 112. One or more machine-learning models may be previously calculated and stored within expert database 120 to allow for rapid selection and generation of a therapeutic corrector 136.

With continued reference to FIG. 13, at step 1320 a processor 104 generates a therapeutic corrector 136 utilizing a machine-learning process and the first advisory input 112 wherein the therapeutic corrector 136 includes a response to a constitutional inquiry. Therapeutic corrector 136 may include any of the therapeutic corrector 136 as described above in reference to FIGS. 1-12. For example, a first advisory input 112 may include an inquiry as to possible diagnoses based on a user's symptoms. A processor 104 may generate a therapeutic corrector 136 that contains a response to the first advisory input 112 that include a list of possible diagnoses. In yet another non-limiting example, a first advisory input 112 that contains an inquiry regarding possible treatment options for a rare disease may be utilized to generate a therapeutic corrector 136 that includes a list of possible treatment options for the rare disease.

With continued reference to FIG. 13, a processor 104 may generate a therapeutic corrector 136 utilizing supervised and/or unsupervised machine-learning processes. For example, a processor 104 may generate a therapeutic corrector 136 utilizing a supervised machine-learning algorithm. A processor 104 may receive therapeutic training data from expert database 120 that includes a plurality of data entries containing constitutional inquires correlated to therapeutic corrector 136. Therapeutic training data may include any of the training data as described above. A processor 104 may generate using a supervised machine-learning algorithm a therapeutic model that outputs a therapeutic corrector 136 utilizing the therapeutic training data and the first advisory input 112 containing a constitutional inquiry. Therapeutic model may include any machine learning process and may include linear or polynomial regression algorithms. Therapeutic model may include one or more equations. Therapeutic model may include a set of instructions utilized to generate outputs based on inputs derived using a machine-learning algorithm and the like. In yet another non-limiting example, a processor 104 may generate a therapeutic corrector 136 utilizing one or more unsupervised machine-learning processes. Processor 104 may receive a plurality of unclassified data entries from expert database 120. Unclassified data entries may include any of the unclassified data entries as described above in reference to FIGS. 1-12. Processor 104 generates using an unsupervised machine-learning algorithm an unsupervised model that outputs a therapeutic corrector 136 utilizing the plurality of unclassified data entries and a first advisory input 112 containing a constitutional inquiry. Unsupervised model may include any machine learning process and may include linear or polynomial regression algorithms. Unsupervised model may include one or more equations. Unsupervised model may include a set of instructions utilized to generate outputs based on inputs derived using a machine-learning algorithm and the like. A processor 104 may generate a therapeutic corrector 136 utilizing lazy learning processes including any of the lazy-learning processes as described above in reference to FIGS. 1-12.

With continued reference to FIG. 13, at step 1325 a processor 104 displays a therapeutic corrector 136 on a graphical user interface 128 located on a processor 104. Processor 104 may display a therapeutic corrector 136 on a graphical user interface 128 utilizing any methodology as described herein.

With continued reference to FIG. 13, at step 1330 a processor 104 receives a second advisory input 144 from an advisor client device operated by an informed advisor wherein the second advisory input 144 contains a therapeutic corrector implementation response. A processor 104 receives a second advisory input 144 utilizing any network methodology as described herein. Second advisory input 144 includes any of the second advisory input 144 as described above in reference to FIGS. 1-12. Second advisory input 144 includes a therapeutic corrector implementation response. Therapeutic corrector implementation response includes any of the therapeutic corrector implementation responses as described above in reference to FIGS. 1-12. Therapeutic corrector implementation response may include an informed advisor's experience with implementing or not implementing a particular therapeutic corrector 136. For example, a therapeutic corrector implementation response may include a description of a particular reaction a user had when taking a particular medication recommended in a therapeutic corrector 136. In yet another non-limiting example, a therapeutic corrector implementation response may include a description as to whether suggested lab tests contained within a therapeutic corrector 136 helped an informed advisor diagnose or not diagnose a particular medical condition.

With continued reference to FIG. 13, at step 1335 a processor 104 receives from an expert database 120 located on a processor 104 a best practices training set 152 wherein the best practices training set 152 correlates a therapeutic corrector 136 to therapeutic corrector implementation responses. Best practices training set 152 may include any of the training data as described above in reference to FIG. 1.

With continued reference to FIG. 13, at step 1340 a processor 104 calculates an optimal vector output for a therapeutic corrector 136 received from a constitutional generator module 108 utilizing a k-nearest neighbor algorithm 156 and a best practices training set 152. Optimal vector output includes any of the optimal vector outputs as described above in reference to FIGS. 1-12. Optimal vector output may be generated utilizing a k-nearest neighbor algorithm 156 which may include any of the k-nearest neighbor algorithm 156 as described above in reference to 8.

With continued reference to FIG. 13, at step 1345 a processor 104 generates an optimal vector output containing an expected therapeutic corrector implementation response 160.

Expected therapeutic corrector implementation response 160 includes any probable or predictable response to implementing a particular therapeutic corrector 136. Probable or predictable response may be known based on currently available medical literature, case studies, journal articles, expert input 116, data aggregations from surveyed responses, and the like. For instance and without limitation, an expected therapeutic corrector implementation response 160 may include a list of expected side effects a user may experience upon taking a particular supplement or medication. In yet another non-limiting example, an expected therapeutic corrector implementation response 160 may include a list of lab values that may be affective either positively or negatively upon initiating a particular exercise regimen. In yet another non-limiting example, an expected therapeutic corrector implementation response 160 may include a list of conditions that a particular supplement has studied indications to be utilized for.

With continued reference to FIG. 13, at step 1350 a processor 104 authenticates a second advisory input 144 containing a therapeutic corrector implementation response as a function of an expected therapeutic corrector implementation response 160. Authenticating may include evaluating by a processor 104 to determine if a therapeutic corrector implementation response matches an expected therapeutic corrector implementation response 160. For example, a processor 104 may authenticate a therapeutic corrector implementation response that contains an adverse reaction that a user experienced upon consuming a particular homeopathic medication to an expected therapeutic corrector implementation response 160 that lists the adverse reaction experienced by the user. Therapeutic corrector implementation responses that may match to one or more expected therapeutic corrector implementation response 160 may be authenticated utilizing other methods. This may include obtaining a second informed advisor response. A processor 104 may display a second advisory input 144 containing a therapeutic corrector implementation response and an expected therapeutic corrector implementation response 160 on a graphical user interface 128 located on the processor 104 to a second informed advisor. Second informed advisor may include any of the second informed advisors as described above in reference to FIGS. 1-12. A processor 104 may receive a second expected therapeutic corrector implementation response 160 from a second informed advisor and authenticate a second advisory input 144 containing a therapeutic corrector implementation response as a function of a second expected therapeutic corrector implementation response 160. This may be performed utilizing any of the methods as described above in more detail in reference to FIG. 7. A processor 104 may authenticate a second informed advisor by authenticating a second informed advisor's credentials. A processor 104 may receive a second expert credential validator, compare the second expert credential validator to a list of known expert credentials stored in expert database 120 and determine that the second expert credential validator is authentic. A processor 104 may authenticate a second advisory input 144 containing a therapeutic corrector implementation response utilizing expert periodical submissions. A processor 104 may retrieve an expert periodical submission contained within expert database 120. A processor 104 may locate an expected therapeutic corrector implementation response 160 contained within an expert periodical submission. This may be performed utilizing any of the methodologies as described above in reference to FIGS. 1-12. A processor 104 may compare a therapeutic corrector implementation response to a second expected therapeutic corrector implementation response 160 contained within an expert periodical submission. A processor 104 may confirm the legitimacy of a first therapeutic implementation response upon confirming that a therapeutic corrector implementation response is contained within an expert periodical submission. This may be performed utilizing any of the methodologies as described above in reference to FIGS. 1-12. A processor 104 may authenticate an advisory input by utilizing user constitutional data. A processor 104 may retrieve an element of user constitutional data from a user database 164. User constitutional data may include any of the user constitutional data as described above in reference to FIGS. 1-7. A processor 104 compares an element of user constitutional data to a therapeutic corrector implementation response and authenticates a first therapeutic implementation response as a function of the element of user constitutional data. This may be performed utilizing any of the methods as described above in reference to FIGS. 1-12.

With continued reference to FIG. 13, at step 1355 a processor 104 updates a best practices module 148 as a function of authenticating a first advisory input 112 containing a therapeutic corrector implementation response. Updating the best practices module 148 may include incorporating a therapeutic corrector implementation response into the best practices module 148. A therapeutic corrector implementation response may be incorporated into best practices module 148 by incorporating a therapeutic corrector 136 and/or a therapeutic corrector implementation response into one or more best practices training set 152. A therapeutic corrector implementation response may be incorporated into the best practices module 148 by incorporating a therapeutic corrector 136 and a therapeutic corrector implementation response into a machine-learning model stored within expert database 120. This may be performed utilizing any of the methodologies as described above in reference to FIGS. 1-12.

Now referring to FIG. 14, an exemplary embodiment 1400 of a method of calculating an inference model is illustrated. At step 1405 a processor 104 receives a first advisory input 112 containing a constitutional inquiry and a user identifier from an advisor client device operated by an informed advisor. Processor 104 includes any of the processor 104 as described above, in reference to FIGS. 1-13. First advisory input 112 includes any of the first advisory input as described above, in reference to FIGS. 1-13. A first advisory input 112 contains a constitutional inquiry. Constitutional inquiry may include any of the constitutional inquires as described above in reference to FIGS. 1-13. An informed advisor includes any of the informed advisor as described above, in reference to FIGS. 1-9. User identifier includes any of the user identifier as described above, in reference to FIGS. 1-13.

With continued reference to FIG. 14, at step 1410, processor 104 retrieves an expert input 116 from an expert database 120 operating on processor 104 as a function of first advisory input 112 and a user identifier. Expert input 116, includes any expert input 116 as described above in reference to FIGS. 1-13. Expert database 120 includes any of the expert database 120 as described above, in reference to FIGS. 1-13.

With continued reference to FIG. 14, at step 1415, processor 104 selects a machine-learning process as a function of an expert input 116. A machine-learning process includes any of the machine-learning process as described above, in reference to FIGS. 1-13. Machine-learning processes may include supervised machine-learning processes, unsupervised machine-learning processes, lazy-learning processes and the like.

With continued reference to FIG. 14, at step 1420, processor 104 generates a therapeutic corrector 136 utilizing machine-learning process and the first advisory input 112 wherein therapeutic corrector 136 includes a response to a constitutional inquiry. Therapeutic corrector 136 includes any of the therapeutic corrector 136 as described above, in reference to FIGS. 1-13. Constitutional inquiry includes any of the constitutional inquiry as described above, in reference to FIGS. 1-13.

With continued reference to FIG. 14, at step 1425, processor 104 displays a therapeutic corrector 136 on a graphical user interface 128 located on a processor 104. Graphical user interface 128 includes any of the graphical user interface 128 as describe above, in reference to FIGS. 1-13.

With continued reference to FIG. 14, at step 1430, processor 104 obtains an adherence input 204 from an advisor client device 132 operating by the informed advisor. Adherence input 204 includes any of the adherence input 204 as described above, in reference to FIGS. 1-13. Advisor client device 132 includes any of the advisor client device 132 as described above, in reference to FIGS. 1-13.

Still referring to FIG. 14, at step 1435, processor 104 calculates an inference model 208 as a function of adherence input 204. Inference model 208 includes any of the inference model 208 as described above, in reference to FIGS. 1-13.

Still referring to FIG. 14, at step 1440, processor 104 updates expert database 120 as a function of inference model 208 and therapeutic corrector 136 in reference to FIGS. 1-13.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

FIG. 15 shows a diagrammatic representation of one embodiment of a computing device in the exemplary form of a computer system 1500 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1500 includes a processor 1504 and a memory 1508 that communicate with each other, and with other components, via a bus 1512. Bus 1512 may include any of several types of bus structures including, but not limited to, a memory bus, a memory controller, a peripheral bus, a local bus, and any combinations thereof, using any of a variety of bus architectures.

Memory 1508 may include various components (e.g., machine-readable media) including, but not limited to, a random-access memory component, a read only component, and any combinations thereof. In one example, a basic input/output system 1516 (BIOS), including basic routines that help to transfer information between elements within computer system 1500, such as during start-up, may be stored in memory 1508. Memory 1508 may also include (e.g., stored on one or more machine-readable media) instructions (e.g., software) 1520 embodying any one or more of the aspects and/or methodologies of the present disclosure. In another example, memory may further include any number of program modules including, but not limited to, an operating system, one or more application programs, other program modules, program data, and any combinations thereof.

Computer system 1500 may also include a storage device 1524. Examples of a storage device (e.g., storage device 1524) include, but are not limited to, a hard disk drive, a magnetic disk drive, an optical disc drive in combination with an optical medium, a solid-state memory device, and any combinations thereof. Storage device 1524 may be connected to bus 1512 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1524 (or one or more components thereof) may be removably interfaced with computer system 1500 (e.g., via an external port connector (not shown)). Particularly, storage device 1524 and an associated machine-readable medium 1528 may provide nonvolatile and/or volatile storage of machine-readable instructions, data structures, program modules, and/or other data for computer system 1500. In one example, software 1520 may reside, completely or partially, within machine-readable medium 1528. In another example, software 1520 may reside, completely or partially, within processor 1504.

Computer system 1500 may also include an input device 1532. In one example, a user of computer system 1500 may enter commands and/or other information into computer system 1500 via input device 1532. Examples of an input device 1532 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1532 may be interfaced to bus 1512 via any of a variety of interfaces (not shown) including, but not limited to, a serial interface, a parallel interface, a game port, a USB interface, a FIREWIRE interface, a direct interface to bus 1512, and any combinations thereof. Input device 1532 may include a touch screen interface that may be a part of or separate from display 1536, discussed further below. Input device 1532 may be utilized as a user selection device for selecting one or more graphical representations in a graphical interface as described above.

A user may also input commands and/or other information to computer system 1500 via storage device 1524 (e.g., a removable disk drive, a flash drive, etc.) and/or network interface device 1540. A network interface device, such as network interface device 1540, may be utilized for connecting computer system 1500 to one or more of a variety of networks, such as network 1544, and one or more remote devices 1548 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1544, may employ a wired and/or a wireless mode of communication. In general, any network topology may be used. Information (e.g., data, software 1520, etc.) may be communicated to and/or from computer system via network interface device 1540.

Computer system 1500 may further include a video display adapter 1552 for communicating a displayable image to a display device, such as display 1536. Examples of a display device include, but are not limited to, a liquid crystal display (LCD), a cathode ray tube (CRT), a plasma display, a light emitting diode (LED) display, and any combinations thereof. Display adapter 1552 and display 1536 may be utilized in combination with processor 1504 to provide graphical representations of aspects of the present disclosure. In addition to a display device, computer system 1500 may include one or more other peripheral output devices including, but not limited to, an audio speaker, a printer, and any combinations thereof. Such peripheral output devices may be connected to bus 1512 via a peripheral interface 1556. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1-20. (canceled)

21. A system for confirming an advisory interaction with an artificial intelligence platform, the system comprising:
at least a processor; and
a memory communicatively connected to the at least a processor, wherein the memory contains instructions configuring the at least a processor to:
receive an advisory input comprising a constitutional inquiry;
generate, using a large language model (LLM) and one or more machine-learning modules, a first therapeutic corrector as a function of the advisory input, wherein the LLM has been trained with first LLM training data comprising correlations between keywords;
receive a feedback input in response to an implementation of the first therapeutic corrector, wherein at least a part of the feedback input indicates that the first therapeutic corrector is incorrect;
generate a feedback quality score as a function of the feedback input using a scoring machine-learning model of the one or more machine-learning modules that has been trained with scoring training data including exemplary feedback inputs correlated to exemplary feedback quality scores;
update the LLM and the one or more machine-learning modules as a function of at least in part on the feedback quality score, wherein updating comprises:
generating second LLM training data comprising the first LLM training data and therapeutic correctors that are incorrectly generated using the first LLM training data; and
generating a second therapeutic corrector using the updated LLM that is retrained with the second LLM training data; and
generate a graphical user interface displaying the second therapeutic corrector.

22. The system of claim 21, wherein the LLM has been:
generally trained with general training sets of the first LLM training data, wherein the general training sets comprises correlations between one or more linguistic terms associated with a particular data domain; and
specifically trained with specific training sets of the first LLM training data, wherein the specific training sets comprises exemplary advisory inputs correlated to exemplary therapeutic correctors.

23. The system of claim 22, wherein updating the LLM comprises modifying the specific training sets by:
generating a synthetic data pair incorporating the advisory input and the first therapeutic corrector with the feedback quality score lower than a score threshold; and
augmenting the specific training sets with the synthetic data pair, wherein augmenting the specific training sets comprises re-weighting one or more existing data pairs in the specific training sets that comprise correlations related to correlations in the synthetic data pair based on the feedback quality score.

24. The system of claim 22, wherein updating the LLM comprises modifying the general training sets by:
selecting one data domain from a plurality of data domains as a function of the advisory input and the feedback quality score; and
modifying the general training sets to comprise one or more linguistic terms comprising the selected data domain.

25. The system of claim 24, wherein selecting the one data domain comprises extracting the one or more linguistic terms associated with the selected data domain from a plurality of data sources using a web crawling module.

26. The system of claim 22, wherein updating the LLM comprises modifying the specific training sets by:
selecting one data cohort as a function of the advisory input; and
modifying the specific training sets as a function of the selected data cohort.

27. The system of claim 21, wherein generating the first therapeutic corrector comprises determining at least a treatment using a first machine-learning module of the one or more machine-learning modules that has been trained with first training data comprising exemplary advisory inputs correlated to exemplary treatments.

28. The system of claim 21, wherein generating the first therapeutic corrector comprises determining at least a diagnosis using a second machine-learning module of the one or more machine-learning modules that has been trained with second training data comprising exemplary advisory inputs correlated to exemplary diagnoses.

29. The system of claim 21, wherein generating the first therapeutic corrector comprises determining at least a lab work using a third machine-learning module of the one or more machine-learning modules that has been trained with third training data comprising exemplary advisory inputs correlated to exemplary lab works.

30. The system of claim 21, wherein generating the feedback quality score comprises:
receiving an adherence input from a monitoring device; and
generating the feedback quality score as a function of the feedback input and the adherence input.

31. A method for confirming an advisory interaction with an artificial intelligence platform, the method comprising:
receiving, using at least a processor, an advisory input comprising a constitutional inquiry;
generating, using the at least a processor, a first therapeutic corrector as a function of the advisory input using a large language model (LLM) and one or more machine-learning modules, wherein the LLM has been trained with first LLM training data comprising correlations between keywords;
receiving, using the at least a processor, a feedback input in response to an implementation of the first therapeutic corrector, wherein at least a part of the feedback input indicates that the first therapeutic corrector is incorrect;
generating, using the at least a processor, a feedback quality score as a function of the feedback input using a scoring machine-learning model of the one or more machine-learning modules that has been trained with scoring training data including exemplary feedback inputs correlated to exemplary feedback quality scores;
updating, using the at least a processor, the LLM and the one or more machine-learning modules as a function of at least in part on the feedback quality score, wherein updating comprises:
generating second LLM training data comprising the first LLM training data and therapeutic correctors that are incorrectly generated using the first LLM training data; and
generating a second therapeutic corrector using the updated LLM that is retrained with the second LLM training data; and
generating, using the at least a processor, a graphical user interface displaying the second therapeutic corrector.

32. The method of claim 31, wherein the LLM has been:
generally trained with general training sets of the first LLM training data, wherein the general training sets comprises correlations between one or more linguistic terms associated with a particular data domain; and
specifically trained with specific training sets of the first LLM training data, wherein the specific training sets comprises exemplary advisory inputs correlated to exemplary therapeutic correctors.

33. The method of claim 32, wherein updating the LLM comprises modifying the specific training sets by:
generating a synthetic data pair incorporating the advisory input and the first therapeutic corrector with the feedback quality score lower than a score threshold; and
augmenting the specific training sets with the synthetic data pair, wherein augmenting the specific training sets comprises re-weighting one or more existing data pairs in the specific training sets that comprise correlations related to correlations in the synthetic data pair based on the feedback quality score.

34. The method of claim 32, wherein updating the LLM comprises modifying the general training sets by:
selecting one data domain from a plurality of data domains as a function of the advisory input and the feedback quality score; and
modifying the general training sets to comprise one or more linguistic terms comprising the selected data domain.

35. The method of claim 34, wherein selecting the one data domain comprises extracting the one or more linguistic terms associated with the selected data domain from a plurality of data sources using a web crawling module.

36. The method of claim 32, wherein updating the LLM comprises modifying the specific training sets by:
selecting one data cohort as a function of the advisory input; and
modifying the specific training sets as a function of the selected data cohort.

37. The method of claim 31, wherein generating the first therapeutic corrector comprises determining at least a treatment using a first machine-learning module of the one or more machine-learning modules that has been trained with first training data comprising exemplary advisory inputs correlated to exemplary treatments.

38. The method of claim 31, wherein generating the first therapeutic corrector comprises determining at least a diagnosis using a second machine-learning module of the one or more machine-learning modules that has been trained with second training data comprising exemplary advisory inputs correlated to exemplary diagnoses.

39. The method of claim 31, wherein generating the first therapeutic corrector comprises determining at least a lab work using a third machine-learning module of the one or more machine-learning modules that has been trained with third training data comprising exemplary advisory inputs correlated to exemplary lab works.

40. The method of claim 31, wherein generating the feedback quality score comprises:
receiving an adherence input from a monitoring device; and
generating the feedback quality score as a function of the feedback input and the adherence input.
